SEQUENCE LISTING (29 sequences)

SEQ ID NO: 1 - DNA, 1526 bp, Homo Sapiens
atggcttctaagaagttgggtgcagattttcatgggactttcagttaccttgatgatgtcccatttaagacaggagacaaattcaaaacaccagctaaagttggtctacctattggcttctccttgcctgattgtttgcaggttgtcagagaagtacagtatgacttctctttggaaaagaaaaccattgagtgggctgaagagattaagaaaatcgaagaagccgagcgggaagcagagcgcaaaattgcggaagcagaagctaaagtgaattctaagagtggcccagagggcgatagcaaaatgagcttctccaagactcacagtacagccacaatgccacctcctattaaccccatcctcgccagcttgcagcacaacagcatcctcacaccaactcgggtcagcagtagtgccacgaaacagaaagttctcagcccacctcacataaaggcggatttcaatcttgctgactttgagtgtgaagaagacccatttgataatctggagttaaaaactattgatgagaaggaagagctgagaaatattctggtaggaaccactggacccattatggctcagttattggacaataacttgcccaggggaggctctgggtctgtgttacaggatgaggaggtcctggcatccttggaacgggcaaccctagatttcaagcctcttcataaacccaatggctttataaccttaccacagttgggcaactgtgaaaagatgtcactgtcttccaaagtgtccctcccccctatacctgcagtaagcaatatcaaatccctgtctttccccaaacttgactctgatgacagcaatcagaagacagccaagctggcgagcactttccatagcacatcctgcctccgcaatggcacgttccagaattccctaaagccttccacccaaagcagtgccagtgagctcaatgggcatcacactcttgggctttcagctttgaacttggacagtggcacagagatgccagccctgacatcctcccagatgccttccctctctgttttgtctgtgtgcacagaggaatcatcacctccaaatactggtcccacggtcacccctcctaatttctcagtgtcacaagtgcccaacatgcccagctgtccccaggcctattctgaactgcagatgctgtcccccagcgagcggcagtgtgtggagacggtggtcaacatgggctactcgtacgagtgtgtcctcagagccatgaagaagaaaggagagaatattgagcagattctcgactatctctttgcacatggacagctttgtgagaagggcttcgaccctcttttagtggaagaggctctggaaatgcaccagtgttcagaagaaaagatgatggagtttcttcagttaatgagcaaatttaaggagatgggctttgagctgaaagacattaaggaagttttgctattacacaacaatgaccaggacaatgctttggaagacctcatggctcgggcaggagccagctgagaccaggccctgcctag

SEQ ID NO: 2 - protein, 502 aa, Homo Sapiens [sequence omitted]

SEQ ID NO: 3 - DNA, 75511 bp, Homo Sapiens
tccagcagacatagaaagacacagaaggccagcgccatggctcacgcctacaatcccagcaatttgggagactgaggcttgagcccagaagtttaccaccagcctgggcaacatggtgagaccccgtctctccatatatatatataaaataataaaaattatccgggcgtgttggcatgtggctatagtccaagctactcagaaggctgaggtgggaggatcacttgagcccgacaggtggaggttgcagggttgcagtgcgccaacatcgcgccactgcactccagcctaggcgacagagcgagaccctgtctcaaaaaaacaaaaaacaccccaaacagaaacaacaacaacaacaaaaacccccacagaaggcagtgaaggcttgggataggattacctggtttaaacccatagaatgtcagaattgaaggaagacttagaggtcacctttgtttttttttcttttgatacagagtcgcgttccgttgcccacctggagtgcagtggcgcgatcacggctaccgcagcctcgacctcccgggctcaagcgaccctcctgcctcggcctctcaaagtgctggagttacaggcgtgagccactgcgcccggcctagaagtcacctttcaacttagtcctctatacatgggaaaagtgaggtccagaaagggtatgagttgtaccaggtcgctgaccctgaacagaagctagatgtcttaattcccaaataggactcgctgctaactctttgggcataccttcgcgattcaaggctcttgggtttcccgctttggccgcctcgccaaacccaagtcttacactcttagtgaccccctcctttcctccttcccccttaggtcagcagggtttactcattccaattgcaatgctccatgttgccggagtagctcctcgctagaagagagattagttatgtcaccgtcggctcctcccattggtccgggcgtgtggagcgcggcgccatgacgtcagacgcccaaatgagtggggcggtgaggggaaggaggagggaagtaggacttcaacatggcggctgcggcactggcggtggctacggtgacggcctggcccggagcgggcagagttggaggtggtggcgttcgctctccctaggggctgtcgggagctcagcggggaccgagcctgggaggccggccggtgccagcacctttcggcttctgagacggcggcagcagcggcattcaggtgaggggggcctccctctgggggagggggaagaggggcggagttgggggaggggggctggtactgggagactgggaaacgggatggggggccgagcgaggtgaagggcgccggagctaggaggtgggagggaaaagtggccggagatccgcgattcggggaggattctgggagaagggtcggagtgaggggaccggagttgaaggggcggaggggaagtgggatgcggaaaaatgagttcggattaaggagactaataccctactggaagacaggcatagtagacatctatcgggctggactgagaactcggtgggaactagaggatgtcagggagggggacccagattttagtaaagaaggtgagtggtggggagttgagggtgagctggggacgtgcggtgtagatttagttctccctgaggcaatgagattggggagaaaaggtggagactagaccagaggtgagtgatctttggggatatctttggccttgtctggtcttctccaggtatgtttatccgccttttttgtttcttgggtggaggcttacctatacagtccacctgggcctcaggaaccgccccagtctttcttgtggaacgtcaggttcatttatttgagctatatcagtgatctttgacctttgttgccaaatgtaattgtttccagactagcgtcttaaaagtatggttgggcataaaaatacgttttatgttaaactgaaaaaaaaattgttacttttagtcaccatgcagtgttgcgaaaccagatttggtaatttagttaagtattacgtatattctgttctaactttactcgttagtgctttgtctcttcagttattagcatttgagaatttgaatagatatgatggatgttttcctttttaattttgtcagatttgatggtgggaaaaacaagatacttagtctttagtgatatatagttctttctagttaatgtcccaacctaaactaaaagtttccgtattggccgggcgctgtggctcacgtctgaaatcccagcactttgggaggccgaggcgggcggatcacgaggtcaagagttcaagaccagcctgaccaacatgttgaaaccctttctctactaaaaagccagccgggcgtggtggcaggcgcctgtaatcccagctactcgggtggccgaggcaggagaatggcttgaacctgggaggcggaggtagcagtgagcctagatcgctgcattgcactccagcctgggcaacagagcgagactccgactcaaaaaaaaaaaaaaagtttacgtattatggcaaagaatgtgaaatgtgtcaagtcttaaaagaattacattgtccgcattaacttactataagtgaaattttattaaatttttgtttctagttctattaattcatggtgtttattaaatagctactgtgtgttgcaataataagagtcattataattacagaaatgctaaacttaatgtttctttagtgagtgaccatcgctgtcattgcacagcagataactttactaacctgctagaacctcattggaaaaaaaacttttttttttttttgtttctttttatttttttccagagggagtctcggtctgttgcccaggctggagtgcagtggcgcgatcttggcttgctgcaacctccgcctcctgggttcaagtgactgtcctgcctcagcctcccgagtagctgagattatagacacccaccactacggctggctaatttttgtattttagtagagacggggtttcaccatgttggtcaggctggtctcgaacttctgacctcaaataatcctccggccttggtctccgaaagtgctgggattacaggcgtgagccaccgcacccggcctgttttctttttttttttttttttgagacagagtttcgctctgtgcccaggctggagtgcagtggcgcaatctcagctcactgcaagctccgcctcccgggcttgccattctcctgcctcagcctcctcagtagctgcgattacaggcgcctgccacacacctggctaattttttaaaatatttttttagtagagacggggtttcaccgtgttagccaggatggtgtccatctgctgacctcgtgatccacccgtcttggcctcccagagtgctgggattacaggcgtgagccaccgcgcccggccttttttatttttatttgtattttattttattttttgagacggagtctccctctgtcgcccaggctggagtgcagtggcgcgatctcggctctctgcaagctctgcctcccgcgttcacgccattctcctgcctcagcctcctgagtagctgggactacgggctcccgccaccacgcccggctaattttttgtatttttagtagagacggggtttcactgggttagccaggatggtctcgatctcctgacctcgtgatccgcccgccttggcctcccaaagtgctgggattacaggcgtgagccaccgtgccaggcctttttttttttttttttaatgaaggcaaaggaaaaaaattaaactctgtaaatatgaaaacttcttcagtttctttcgtttagtatttaagactgatttcctaatcatggcatttcttctgtgtattcttctgaaatattttttcattttgattttgaagacagagtattctgaaaagataataatagatctgtagacaagtctcatcaatgattgttagtgcattttttaattaaaatttttttttttgtagagaaggggtctcttatgttgcccagtctgattttgaactcctgagctcaagtgatcctcccaccacagcctcccaaactgctgggaggcatgagcagctagtgcacgtatttttgactggtgaatgtcacattatttggtgtatcttttaactatggtatttaaggatccctgacgtttatttatttatttatttatttatttatttatttatttattgagactgagtctcgcactgttgcccaggctggagtgctgtggcgtgatctcagctcactgcaacctccatttcccaggttcaagtgattctcctgcctcagcctcccaagtagctgggattacaggcacccaccaccacacccggctaatttttatagttttagtagcgacggggtttcacaacgttggccaggctggtcttgaactcctgacctcaaatgacctacccgcctcggcctcccaaagtgcagggattacagacgtgagccatggcgcccagatggatccctgacgtttaattcagtgactaaaattcttattattttcagcattatagttgaaatatcattagccttgagtgtaaatgccaatattacaagcttttcgtgttcttttttctttttctttctttccttctttctttctttctttctttctttctttctttctttctttctttctttctttctttctttctttctctctctctttcttttcttttctttctctctctttctctctctcctttctttcttttcactcagtcgcccaggctggggtacagtggctcgatctctgctcactgcaagctccgcctcctgggttcacgccattctcctgcctcagcctcctgaatagctgggactacaggtgcccaccaccacgcctggctaattttttgtagttttagtagagatggggtttcaccgtgttatccaggatggtctcgatctcctggctaattttttgtagttttagtagagatgaggtttcaccgtgttagccaggatggtctcgatttcctgacctcttgatccgcccatctcagccttccaaagtgctgggattataggcgtgagccactgtgcctggcctctgttctttagttatgttattgaatgtattgaaatttttcaaatgtagatcattttaagagtaatcttttttcctacagattcattaaaaatatttattcatcatttatcaggtggttttctttagacagtctttctcaagaaatgcaaaaaggtaatacagagtgccttaaaacattaaataggcctggcgccatggctcatgcctgtaatcccagcactttggggggccgaggcgggcggatcatgaggtcaggagattgagaccatcctggctaacacggtgaaatcccgtctgtactaaaaaatacaaaaaaattagccgggcgtggtggtgtgtgcctgtagtcccagctactcgggaggctgaggcaggagaatggcgtgaacccggccactccagaggcggagcttgcagtgagctgagatagtgccactacactccagcctgggcaacagagcgagactccgtctcaaaaaaaaaaaaaaaacattaaataaaccttttttctaacaattaaaaaaaaagaaaaaaactcaagctgaaaagaagcttagaaatcacctgggtgtagtttcccacacagaatgggaatccctgcttctataccaaaacaaatgaaaatccatcctcttttccagatttctaaggaagtaataggatttttcagaatttctttttttttgtttgttttttgttttttttttttgaaacggagctttgctcttgttgcccaggctggagtgcaatggcgccatctcagctcaccgcaactgccgcctcccgggttcaagtgattctcctaccccaggctcccgagtagctggaattacaggcatgtgccaccatgcccggctaattttgtgtttttagtagagacggggtttctccatgttggtcaggctggtctcaaactcttcaactcaggtgatccatccgcttcggcctcccaaagtgctgggattacaggtgtgagatagcgctcccggcccagaatttcttataggaaaagtatgttcaattagaaaaaagaagcaagcagagtagtgctcttccaacctgtaacatacagtgtcctgtaacattagaaaataacttgaaggtaacagtaatattttcctatgaatcgagagttacgatttcaggttttagaaattttaggtggaagtagcaagattcacaaaggaaagggtgctgacactgggtatgtactaatgagccaggattgttaaacactttctttccttttttttttagatagtctcgctctgggccgggcgcggtggctcaggtctgtaatcccagcactttgggaggccgaggcgggcggatcacaaggtcaggagatcgagaccatcctggctaacacggtgaaaccctgtctctactaaaaatagaaaaaattagccgggcctggtggcgggcacctgtagtcccagctactcgggaggctgaggcaggagaatggcgtgaacccgggaggtggagcttgcagtgagccaagatcacgccactgcactccagcctgggcaatagagcgagactccgtctgaaaaaaaaaaaaaaagagagtctcgctctgttgccaggctggagtgcagtggcacaatctcggctcactgtaacctccacctcccaggttcaagagattctcctgcctcagcctcctgagtagctgggactacagatggtgtgccaccatgtccggctaatttttgtatttttagtagagacaaggtttcaccatgttggccaggatggtcttgatctcttgacctcgtgatcctcccacctcggcctcccaaagtgctgggattacaggcgtgcgccaccgcgcccagccaatcctttttttttttttttttttttttttgagatgaagtctcgctcttattccccaggctggagtgcaatggtgtgatctcggctcactgcaacctctacctcccgagttcaagcgattctcctgtgtcagcctccctagtagctgggattacaggcgcctgccaccacgcctgctaatttttgtatttttagtagagatggtgtttcaccttattcaccaggctggtctcgaacttcggacctcaggtgatctgcctgcctcggcctcccaaagtgctgggattacaggcatgagccactgcactcggccctgttaaaacactttacataatctcagttcttggccagacacagtggctcatgcctgtagtcccagcagtttgggaatcccaggcgggcggattgcttgagcccagaagttcaagaccagcctggacaacacagtgagacctgtctctacagaagtcacagaaaaattagctgggattggtggttcatgcctgtagtcccaggtacacgagaggctgaggtaggaggatcacctgagctcaggaagtcgaggctgcagtgagctgagatcatgcccctgcactccagcctgggcaacagagcaagatcctacctcaaaagaaataatctcgggcgggtgctgtgggtcatgcctgtaatcctggcacttggggaggcccaggcaggtggatcacctgaggtcaggatttcaagaccagtctggccaacacggtgaaaccccgtctctactaaaaagacaaaaattagctgggcgtggtggcaggtgcctgtaatcccagctactcaggaggctgaggcaggagaattgcttgaacctgggaggcggtggtggcagtaagcctagatcatgccactgcactccagcctggctgacagaacgcatctctgtctcaaaaaaagaaaaacaaacaaacaaacacattcagttctcacacttgaattaggtaggtaggttgtgttatgcgtgctttttatgtattgaggaaacaggatcacagaggttgtgtaacttaactaaggttacaactaggtagactgaatttgaactgaaatctgtccaacctcaatgcctatgctgcattcttttatgccattttgcccctcttaggtggtcacaggaccattccaattcctataaggtataagcactagttttaaagtaataccattgtgaactgcttgttctcattgaaataatagtgtaactggaatgataaatagttcattaaaattttagtcacattatgtcatcagaattctaggaggaaatgaatacctgattattttgtacttcacaaaataaactggataaactggataaatgactgaaagtttcttttttatattgctgagtcagattggatccggttcattaagggccaaaatttgtatttggcagtaatcctcatgtgttctcagaatgcttgattggtcattatcagttgcttaatgcttaccaaacacttccagtgttttgagtcactgaattggctcatggtaattgttaatcttgtggttactgagtcaagagccagcagtgtgtgttttggctgcttcacttcctcctcaacacttgatattgtcagattttaaattaattttttttttctaatctggtgggtttgaaattgtatctatagtgttttttatcagcatttcctgaagctgggcatcttttcatgtattttcgttggtcatttgtgcttctgcgaagtgtatattcaatttcgttgcctatttttttggtcttttctctttttctgatttatttttaagagttctttatatattctggatactaattatttgttaatttataggttgcaaatatcctttcccaatttgtgacttgtcttttcatctcttcatagtcttctggtgagaaaagctcctaattgtaatgaactgaatttttcagtctttcatggtttgtgttttgtgtttttttttattttttattttttattttttgagatggagtttcgctcttgttgcccatgcgggagtgcaatggcgtgatcttggctcactgcagcctccgcctcctgggttcaagcaattctcctgtctcagcctcccgagtagctgggattacaggtgcctgccaccacgcccagctaatttctgtatttttagtagagacagggtttcatcatattggtcaggctggtcttgaactcctgacctcaggtgatccacccgcctgggcctcccaaagtgctgggattacaggcgtgagccactgcgcccggctggtttgtgcttgttgtatcaaatttgggaaatccttctttgctgccaggcatgttgttttaataaatacagtagtatacctatctataatgtggtatatctcatctgacatttatttccaatatttgcttgattacaaaggcagacttcttagaagttgctttcttattatctttggagttatagggccacagggttttaaaaggaggttggattgtgccttacatgtgggtttcatacattgtccaaactgagacaccaacagatttgtgaatgagatgtgcatcttattaaatgttaatgtgatttgacaatgagacctttctttgtgtggacagtattatatacaattgatagtgtgtcattcagtctaagctgttagcactttcactaaaatatattcaaattttttttgatgtctagtttttacagtgttgggctttatcaagaatagttgtggattgtggaagagctgatatggaaacagaggaatgtattttgcttttttttttaaactctgtccggttccttggacttgtgtgtctcctatcttatcaagaataattgtagattgtggaagagttgatatggaaacatcttagaagaatgttttttgctttttttaaaaaaaaaaacaaaactgtccagttccttggacttgtgtgtctcctatctaccattaaacccttatgacttaaggtatatgtataaatacatatatacatcttggtggcattgatttttctatgttgtgtattaaagctgctaaaaaacattttttctttttttcaatgagaagactagtgattactttgactttaggaattattcttctaaggagtcaagtatttgatctgggcgaggatcacactgaaatataaaacatctgagtaatttgggttcactaaaaaaaaaaaaaaagaaatgtaaaacaaccaccagattaaaaatacactacaaatactgtttacaaccaccagattaaaaatacaacacaaatactgtttatttatacaaatagtatacctttgacatttatgaaaggcatactcttagatcattgggaatccgtacttatgtaactaccatagtttatagtgtccttcatgaaatacagaaaagtgtaacaggaaatttttagttaccatcagatttgatgggtaaatagaagagagcaacaaagtaaagcaatttgaatgctaaacttagtcttcagcttttttttttttttttttttttttaaagagatgggatcttgccctggtacccaggttggtcttaaatgcctggcctcaagtaattcacctgcttcggcctccccaagtgctgggattacagacgtgagccaccatgcctggtgctttcagctacctttaacttcctgcctttttctgttgtttgttactaccactggcctattccagcctcctttagtaacctattggaaactcaagattagtctgtcttgggccgggtgcgttggctcacgcctgtaatcccagcactttgggaggctgaggcgtgtggatcacctgaggtcaggagttcgagaccagcctggccaacatggtgaaaccctgtctctcctaaaaatagaaaaattagccaggcatggtggtggattcctgtaatcccagctactcaggaggctgaggcaggagaattgcttgaagccgggaggcagaggtttcggtgagccgagatcgtaccattgcactccagcctggccgacaagagtgaaactccatctccaaaaaataaaaaataaaaaagattaggctggctttgcctgtcttttgcttcatctcatcatcatggtcttggtcccaggtggggtttgcatccttactggtaagaatggaatcttcctaatcctaaaatctttctatagtttgggagtgttaacatttccttcagtagtttcatggggaaggatatgggcagtaacttccccatttctttcttctagtagatcattatatcttttttttttttttttaaagacagagtcttgctgtgtcgcctagcttggagtgcagtggtgcgatcttgactcattgcaacctcctcctccagggttcaagtgattctttcacctcagcctcccgagtagctgggattacaggcacgtgccaccatgcctggctaatttttatatttttagtagaaatggggtttcaccatgttggctaggctggtcgcaaactcctgaactcaaatgatccacctgcctcatcctcccaaagtgctgggattacaggcatgagccaccgcacccggccgatcattatgtcttataggatacctcatgagagaagtgatctggctgcatgggtggaattcaaggtcaaatggtcagccaaattaaaaatcactgctggagcagactttaggggtagtttgtgaactttcaaatttggaaattattattattattattattttttgagacggagttttgctcttgttgcccaggctggagtgcaatggtacaatcttggcttactgcagcctctgcctcttgggttcaagcagttctcctgcctcagtctcccgagtagctgggattacgggcgtgctccaccatgctcgattaattttgtatttttagtagagacggtgtttctccgtgttggtctggctggacttgaactcctgacctcaggtgatctacccgcctcagcctcccaaagtgctgggattacaggtgtgagccactgctcctggccagacatatttaattctttcatcaaaggtttattatatttccgagatattctagccaaaggaaacaaacttaacaacttttgttttctgaaaatactttagcaaaaatagaagggagaggatcaggaaaaataactaatggataccaggcttaatacctgggtgatgaaataatctgtacaacaacccccatgacacaagtttacctatgtaacaaaccttcacatgtacccctgaacttaaaagttaaaaaaatttaatgttaataaaatcttaaacaaaaatcacggaaggtctttaattgtttatgtcgtgtcctatccgttttcttgaagaagaaatattagctagttcttgtaatttatttattttttggggtggagtcccactctgtcacccagactggagtgcagtggtgtggtcttggctcactgcaacctctgcctcctgggtccaagcgattctcctgcctcagcctcctgagtagctgggattacaggcccttgccaccatgcctggctaaattttttatttttagtggagatggggtttcaccatgttggccaggctggtcttgaactcctgatctcaggggatccaccttcctcagcttcccaaagtgctgggattacagacttgagccaccatgcctggcagttcttgtaatttaatagggaatcttttttcctttctttctttctttttttttttttttttggagacagggtctcgctctgttgcccaggctggagtgcagtgatgcagtcacagctcactgctgcagccttgacttcctgggttcaggtgattctcctacctcagcctcccgagtagctgggactgcaggctcatgccaccatgcctggctctttttttttttttttttgtattttttgtagagatggggtttcatcatgttgcacaggctggtctcaaactcctaagttcaggtgatccacctgcctcggcctcccaaagtgttggaattacaggtgtgagtcacagtgcccagcctgctctggatatattactggcattatatttagtggaagagcatatatatattagagtaatggagatttatttcatttagtagtataagaaattatgtttaggtgcagttctaatttgagacattctgtatctgtcctgtctgatatgtctccaacattgacagtggagatataaaacattttcatcagtgtagaaagttctgctggacagcactgttgtaggtcagtgattctcaaagttttagtgtgtgtatggataacttggatgtattattaaaatgtagattctgagtcagtagctctggtgtgggcttgagagtttgtatttatttatttatagagatggggtcttgctatgttccctggtctgaaacttctggcctcaagcagtcttcccaccttggcctcctaaagcaccaggattgtgggtgtgatccactgtgcctggcctgtgtaaactttaaaatagtgtctttagccatgagggatgatattgtaatggcagaactctggacagtgagttgaaagatcgaaagttgtagttcactttttccaccatcttgaagcttttagaggcctgctgggaataggacttctaaaaggcaaatatgtctggaaggctgtggtccaaggccattttttgttggctgtaagtgtggtctccagaatcaaagagagcacacaactgtttttaaatcgaaggtgtttatgcctgagatgaagctgaattctgtttgggcaagagatttgtgcttatgtatacaaagcaaagaacaacacagtgactcctggtggcaaaccaaacaaaaccagagtaatctgtggaaagataatttgcgcccatggaaacagacatgccaaattccaaaggaatcttcctgctgaggccgtttggacacagaattcacgtgatgttgtacccctcaaggatttaaactactaaaaagtaaaagtggatttgtgaacaaaaaaagtcctagttcagctttcctgtttaaaaaccaacctctctcagccttggatttttttttttaattttttaaatttaaaaaaaatttttttaattaaatttttttttttagtgatggggttttgctgtgttgcccaggttagtctctaattcctgacctcaagtgatcgtccctccatggcctcctaaagtactgggattacaggcatgagccacggtccccggcctgtataaactttttaaatagtgtcttttgctatgagagcttgtattgtaatggcagaactgtggacagtgagttgaaagatctaaagttcaggccaggcgcggtggctcatgcctgtaatcccagcactttgggaggctgaggtgggtggatcatgaggtcaggaatgtgagaccagcctgaccaacatggtgaaaccccgtctctactgaaaatacaaaaattagccgggtatggtggcgcacgcctgtgatcccagctactcaggaggctgagacaggagaatcacttgaacccgggaggtggaggttgcagtgagccgaggtcacgccactgcactctagcctgggtggatagaatgagactccatctcaaaaaaaaaaaaaaaaaaaaaagatctacacttctagttcatctttccagttttaaaaccaatctctctcagccttgaactctttttttatttttcaattttctttagggatgggggtcttgctatgttgcccagtttggtctcaaactcctggcctcgagcatacctcccaccttggcttcccaaagtgctgggattataggcatgagccaccaagcctggctaatgtatttttttaaaatcacaagttcataatgatatttattatttgtttaacttttagcccctgcaaagaaatataatttagattttagaattataggactttaaatattttgcctttcctcttttatataaaaatattagttctttacaactataattacttatttttctatattggactcatataaaaagggttttaaaatagtaatactgctattatagctgataagtacaatctaaaatctttgctattctttatgttcttaaaatactgtgtaagggagggccaggcgcagtggcccacgtctgtaattcccagaactttgggaggcgaggcaggcagatcacttgaggtcaggagtttgagaacagcttggccaatgtggcaaaaccccatctctctactaaaaatacaaaaaattagccaggcgtggtggtatgcacctagaatcccagttactcaggggggctgaggcaggaggatcgcttgaacccaggaggtggaggttgcagtgagctgagatcacgccactgcgctcccgcctgggtgacagagtgagactctgtctcaaaaaaataaaaataataaaaatattgtgtaggggaagctctcaaaccacgttcttcctctactctcacaccaccacaacaatcatcaacacagaacacatctgtgaccaaaagtgtgggggtttttcacctctgcaccaagcagtggacaccagctcggtgttctccaattcaggttgggggctcagtcttcaagactgcctgcacacccctggacaccagttgcatgtctggggccctggatcttatgacccactgtcttcatgttggtgttcccacgatcccctctttgggtttggttaatttgctggagcatctcacagaactcagggaaacactgaaatttattggtttattctgaagaatcttacaaaggatatagatgaagggatgcatagagcaaggtatgggggaagaggtgtggagcttctatacccttcttggggcaccaccatccaggaacctccaggtgttcagctatccagaagttctctgaacccagtcctcttgggcttttgtggaagcttcatgatgtcaggatgccttctctcagggtatagggcagtcccctctctggggagggtgttaagacccacaatcagaaaggtggggaaagattatagtcctttcttggggcaggtgaaagtagggcagaggaagaggttttgtttcctgagacctgcccctgagacctaacacacccaatattgtatcagaagactgtaactagggctgtgggaattatgaaccaggaacagtggatgaaaaccaatatatgtcataacactacaaatatattttactgatgtatacaattagaataccgagttctaaagtcacttcaaataattatattctctggtcattttactaattttctgtaaagttaggtttatttgtttcattttgtattcattttgagggctaaaaaattatattgctaagaatgtgtaatagctattttgaaaagttagaatacagagatgtataaaaagaaaaattctctctctttttttttttttgagatggagtctcgctctgttgccctggctggagtgcagtggcacaatctcggctcgctgtaaactccgcctcccaggttcacgccattctcctgcctcagcctcctcagcagctgggaccacaggcacctgccaccatgcccggctaattttttgtatttttagtagagatggggtttcactgttttagccaggatggtctcgatctcccgacctcgtgatcctcctgcctcggcctcccaaagtgctgggattataggcgtgagccaccgcacctgaccaataaaaattttctttaagccaccattggaaatatccactattagaatatttatataaaatatttgtggagctcacatttattgaaaacattatgccatgtgttcagattgttctaagcactttgtatgcattatctcatttgatccttacaataactttatgagatagttactattatctctattttatttttcttttttcagccagtcaaatttagcagtggggggttgtataccaactttagtaacactgctttgaataagttctgatgagccactaccattggaccagcctgttattcccatttgattgattaaaactcaaatgttaacttgtacaagggatacgtaacctagtaagttgcagaactgagacttgacccctgacaattcttgctcaaactcactttattcatccctgtgtattgtttttatctttttttctgtgcttacacatacacggatttttttcttttgtcatttgagaacagagattttttaaatgtatttcatacattcaggtaagtttttttttttttctctaacagtgtcttataagaagtttttgatttcattgaagtcccctttagtttttcctttatggtttgtgcttttggtgttatatctaagaatttttgcttaatctaaggtcagaaagatgttctcttcttgaagttttatactttcaggtttgacttttttgtctatgattcattttgaattaattatatatgacgtcatatggatggaagattttttttgtttgcacatagatatctattttagcactttttgttgaaaagattaccctttctaaaatattttgtccatgtctatatttggacctgttctattctattgatctgtttgtctatctttatgccagtatcacactatcttgataattgtagctttataataaattttgaaaccagaaaagaaagtttcccaaagttgttacgactgtagagcctttggatttttcttttttttttttttctgagacggagtttcactcttgttgcccaggctggagtgcaatggcgcgatctcggctcaccgcaacctccgcctcccaggttcaagcgattctcctgcctcagcctcccgagtagctgggattacagacatgtgccactatgcccggctaattttgtatttttagtagagacggggtttctccatgttggtcaggctggtctcgaactcctgacctcaggtgatccacctgcctcggcttcccaaagtgctgggattacaggcatgagccaccacgtccggccagtcctttggatttctatacaaattttttggtttttttttttttttttttttttttttttttttttttgagatagagtctcactatgttgcctaggctggcctccaacttttgggctcaagtgatcctcctacctcagtctcctgagtagctgcaactacacaaacacgctaccatgtctggctccatatgatttttaaaattagcttgccaagtttctataaaaagctgctggctttttatttatttatttatttatttgagaccaggtcttgctctgactcctaggctggagtgcagtggtgtggtcatggctcactgcagcctcgacctttcgggctgaagtttatcctcctacctcagcctcttgagtagctgggaccacaggtgtgtgccactaagcttggctaattaaaaacaatttttttttttttttggagacggaatttcactcttattgcccaggctggagtgcaatggcgtgatctcggctcactgcaacctctgcctcctgggttcaagcgattctcctgcctcagcctcccgagtagctgggattacaggtatgcatcaccacgcccggctaattttttgtatttttagtagagatggggtttcgctgtgttgaccaggctggtctcgaactcctgacctcaggtgatctgccctactcggcctcccaaagtgctgggattacggccacggcgcccagcctaaataattttttttaatatagacacggtctcactatgttgccctggctgatcctgaacttttggcctcaagtgatcctcctgtctcagcctcccaaagcaccgggattacaagcgtgagccaccatgcctagcttcactgattcttcagcttgattgagtctgctgttgaaattctctattgaattatttggttcagtcattatattcttccgctctaagatttgctttttattatttgtttctttgttttcattttgttcatgtattgtctttctaatattgttatttgtgtgtgtgtgtgtgtgtgtgtgtgtgtgtgtgtgtgtttttaattgagatggagtctcactctgtaacccaggctggagtgcagtgacacaatcttggctcactgcaacctccacctcctgggttcaagtgattctcccgcctcagcctcccgaatagctgagactacaggcgtgtgccaccacacccggctaatttttgtatttttagtagagacggggtttcactatgttggccaggctggcgttgaactcttgaccttgtgatccgcttgccttggcctcccaaagtgctggaattacagatgtgagccactgtgcccggccagttatttgtgttttattatagttcacggaccttctttaataggattatttatttatttattattttgtaaaaatttttattaaaattttttttttcttttatagagacgggtttcactatgttgtgctagtctcaaacagccggggctcaagcaatctgcccgccttggcctcccaaagtgctgggattacaagtgagagccactgcacctggccaagaggattattctgaattctttgtcagtcagttctatagatctccatttctttagggttgttttttggcattttattagttttcttttgtggtatcatgtttccttggttctttttttttcttttgagacagatttttgctctgtcatctaggctggagtgcagtggcacgatctcagctcactgtagcctctgcctcctgggttcaagtgattgtcctgcctcagcctccccagtagctggggttacaagcatgtgctaccatgcctggctaatttttgtagttttagtagagatggggtttcaccatgttggccaggcttgtcttgaactcctgacctcaggtgatccacctgcctcagcctcctgaagtgctaggattacaggcgtgagccactacacttggcctccttgattcttaatgatctttttgtccttgtgttggtttctgtgcatttgaggaagtagtcacttcttttagtctttaccaattcatttctgtgaggaaagaccttcaccagtcagcctaacctggggttctggatgggtcagctggtagtatctgtgggcaagcagggcgtgctgctggaatctcttgttgaacttggtagctgcccatactctgaggttaggtggggcttgttctctggggtctgaggccagtgcaggtctgttggctgggctctgaggttaggaggggcagctggctgggctccatgtttaggccaggctgctctgtgacttgggagggctgcaggctgtactctggaatttccccggtcaggtgaggctacagggtatgcaccatagatgggtaggactataggctgcattctgcaatcaggtggggttgtatgttaagtttagctgttgaatgggggcattggctgatttttttgcctggatggggctgctggccctgctccaaggttagacgggttctgtggccgggctgcctggttgggcaggcaggttgcctggttggtcctgtggagttggagtttcagctatactattaggtgaggccataggctatgctcagctgaggtcagactggatcactggcacaggacccaagccaggcagaatgttgactgtgcttgttggatagacatgccgcagatgggcagagctggtggttgggctctctggttgggtgaggccactggcagggatgtggtctcattgtcatgatttgcatactgtttgctgtgagccccacccccttctttgttcctagctgactccagtggtctagccctcccatttcttccctatgaggcaagacagacatggatctcctggaaagcaccctggaaagctgggaaagttggggaagctgaatgtctgccttgggctttctttttcccactgcagaaactataggcccaggggatcactctctggccccatgtgggcctggtgaggggcgatgcagtcaaaaagtcaagccactcttacccgtctaatggggcttttggtatttgtgctccaagggagtgcttaagccctaggctctggaattttcacaaaggtgttcttgtctgtggataattactaattgacatttctgtgatagtgactagaacctggacctcctattccaccatcatgttcttggactcttttttattatttatttatttttttgagacggagcctcgctctgtcgcgcaggctggagtgcagtgatgcgatttcggctcactgcaacctctgcctcccgggttcaagcagttctcctgcctcagcctcccgagtagctgggactacaggggcacaccaccacgcccagctaatattttgtatttttagtagagatggggtttcaccgtattagccaggatggtctcgaacccctgaccttgtgatccccccgccttggcctcccaaagtgctgggattacaggcatgagccaccgcgcccagcctattatttatttttaattttaatttttttgagacactatttctattttggaataattttagatttacagaacatttgtagagatagtacagagagtacagagagttcctggctatccctaactcagttttccctaatgttaaccatcttacattaccatgatacatttgtcaaaactgaaaaacctaccttggtacgttagtatcaactaaactctggactgtatttagatttcccctgtttttctttcttcttcttttttttttctctctctttttttgagacagggtctcattgtgtctccccaggctggagtacagtggcatgatcatgtcctactgcaaccttgactacccctggctcaggtgatcctcctgcctcagcctccctagtagctgggactacaggcacgtgccaccatgcctggctgatttttttttttttttttttgtagagactgggtttcaccatgttgcccaggctggtctggaactcctgggcttaagagatccgatccttctgcctcggccttgcaaagtactgagattacaggtgtgaaccactgcacccaacccaggttttctacaatttttttttttctatttcaggatccaatctaggataactaaattgtgtttagttgttatactttcttagtttcttctggtctgtgatagtttcttagtccttccccccgctcccagtaatttgacagctttggggaaaactggtcaggtcctcaatttggttttgtctgctgttttcctcgtaattagactgggcttgtggatttttggaaaaatacctgaggtaaagtacctgtctcagttatatgaaatcagtatggacttgagtatttagtttatattttggattaaagtcccacattgatttattttgttgctcaagttgttttatctttggccattgagagctctttcagattggccttggccttagaatctgtcatttctccagagaatagggcttgtttctctgtcagtctgatcttcattcctttgtaaagacttcatttgtggtatcttgataacttttaggagccacccttttaggagggggtttgttagtgctattacggttttattttttaagagattatgattaggatggatagatgtttagtttgccaacctgatctaaacttttaatttttgctaaacagaatttgtagcatttagagctatatgctttagaatttttcattgtatgatgtttaacatacagaaaaaatatgaatgtaaaacatctaagttatgaggcataataataaacatttgcaagcttattagttgtcttgaaaactagagcattaccaatacattactatttatttatttgtctgtttctgtcctatcctatccttttactactccacagagttagctgttctcccgaattttgggtttatcatttccttccttttataaaccacatgatatatattcctaaataatatattgtttatttttgcttaattgtgagctttataaaaatgttaaactgtatataggcttaagtgattttatttttttttgagatggagtttcgctcttgttgcccaggctggagtgtgatggcgtgatcttggctcaccgcaatctccgcctcccgggttcaagtgattctcctgcctcagcctcccgagtgagtagctgggattacaggcatgcgccaccatgcctgcctaattttttgtagttttagtggagacgtggtttctccatgttggtcaggctggtctggaactcctgacctcaggtgatccaccagccttggcctcccaaagtgctgggattacaggtgtgagccatggcacccagccttgttttagttctttattttagaaagttccaaactcataaaagtagatttggctgggcatgactcatgcctgtaatcctagcactttgggagggtgaggtggccagatcacctgaagtcaggaagttcgagaccagtctggccaacatggtgaaaccccaactctactaaaaatacaaaaattaacgagtgtggtggtgtgtgcctataatcccagctacttgggatactgaggcaggagaatcacttgaaccccagaggcagaggttgcagtgagctgagatcgtgccactgcagtccagcctgggtggcagactgagactgtcacaacagcaacaacaacaacagcaacaacaaaaatgtagattaatcctgctttgtaaaaactcttatttttttctactcacacaccaccacagcaattatcaacacagaaaaagaattctgtggccaaatgtgtgggcagttttccccacacaccaagcagctgccaccagttgggtgttctccagttcaatgccgacactgtctacctggagatagtgtcagatcccactggttgaatgcgcagtctccaagattgccccctagcactcatcagtcacaagtctgggcctcaagtgcttctgaccaccagcttcactcatgtcagggttccctcggtcctctctttaggtttaatttgccagagtgtttcacagaactcagagaaactcttttatgtttactggtttattctaaaggcgattgcaaagtatacagatgaagagatttgtctggcaaggtaggggggaaggtgcactgactttccatatcctccctggggtgtcaccatccacgaacctccgtccaggtgttcagctagccagaagctctccaaatccagtcctcttgagtttttatggaagcttcatgatgttagcattccttctcccagagtacagagtgggagtctccgtctttttcttttgagacacgctttcactctgttgcccagctggagtgcagtggcaccatcttgggtcactgcaacctccacctccgagtagctgggattacaggcatgcaccaccacgcccggctaatttttgtatttttagtagagatggggttttaccatgttggtaaggctggtctcaaactcctgacctcaagtgatcctcccgcctcagcctcccaaaatgctgggattgtaggagtgagccaccctgccctgcctgtgagaggggcttaagactcacaatgagaaaggggtattgggagagatagagttctaccttaggacagttgaagggagggtgggaaaagatttgagagattctgttttctgaggcctgacatacctagcattataacagaagactccagcaagggctatgggagttatgggccagaagctgtagacagaaacgtgtgtgtgtgtgtgtgtgtgtgtgtgtgtgtgtgtgtgtgtgtgtgtgtgtgtccccgtccccgtatatgtatgtatcataacaccatacagccccaataactgtcaatcttgttttatatacataccctctgcccagttattttagagcaaatcccagtcatatcatttaacctgtcaagttatttttgtatatctctgaaagatcacataaattactataatagcattatcctacctatagaaaatgaacttattccttaatattatcaaatattcatgttttctcaatttttttattgtgttttcctcatgtgtttggattgggactcaaataaggtctgtaagttagtgattgcttgagatgtattccttaagatctttttcagtctgtaggttctcttttcatcttgctttttttgtgttgaaggaaactgaatcactttttctaaaagtttgctacagtcaggattttgcagtttaattcactactattttaattaggatttgggtatgtatggataaaacataaataatcagctaggtgtggtggtacattcctatagtcccagatacttgggaggctgaggcaggaagatcttttgggctcaggagtttgaggctgcagtgagcaatgttcatgccacttcactccaacctgggctgttcagagcaagaccctgtctctaaaaacaaggatcaacaaaaattaaatatgctttcatttctggttcttttttctcttttctctgtcagtttttggtatcagtgttatgctgatctcatacgagaacttggaattcagcaacattcattcagcaggagttactgagcactactgtggccgggcactatgcttggtgtaatggtgaatgagacagaagtggtatttgtcctcgtagtgcataaaggcaagtggggaagacagacaataaacaaatcctgaataaattgataagctacaaatatatatttgtttccattttatgtgttggaatagattaaataaaataggaataatctgtttcttaaatatttaatagaactttaaagacatctgttctgacatttattttagcatacacatatttcataagcatgtgaaattacttaagaaccccttttctctccccataaatctttgacaccgtgtgaagttgttccatccttatattgtttttcctttaggcctacagtgttcccctggggatttgagttttcaagttttgtgaacaaggctgtattatttctgtgggaaaaatcattcaggtttcacatgatggaaacagaatgatacaatccactgagtttgaagactccaggaagaacaaaaaattatatttgcaattacttatttcaggtcaaattaatagcttctagtatgataacttttttttgttttaaaattaaccagttaagagactgggcgtggtggttcatgcctgcaatcccagagctttaggaggccacagtgggtggatcagttgaggtcaggagtttgagaccagcctaggcaacgtggtgaaatcctgtctctactaaaaatacaaaaattagctgggtgtgatggcccacgcctgtaatcccagctactcgggaggctaaggcacaagaatcttgctgctgcactccagagtgcagacgacagagcaagactctgtctcaagacaacaacaacaaaaaactgaccagttagtattgtattagttacttgtagaggaaaatagtatcaaatagtaaggattttcttataaaaaacaaaaaaagggtgtggattgtatatacttgctgagtcaagtatacttgtctttctgagtcaagtaagtatacacagtccacacccatttttttttttgaagaaaacacccgtttgtttttatgtgccctcagaattaagatgaagtcttgtgctatctttttaatatgtataagactagaaaaaccagtgaaattcatagttagaaaataatctatctctagtcctttaattctggaaagaattccagagatcatttactccagtagtagtcttgttttatggaagaagccctaaaagttctttttttttaaatttcagaggtttttacccattctacaagtcttttaattgcctcaggtgctcttgtctttcttgccttctggtatgaattgtgatatgatttttggagttgggaaggaaattatatttttggttcaataacttgttgacattatttttttgccaaccagtctttattcagttgcaagaattaaattgataggttcgtggtcagatttcctcttgaggacactgcactgaactctctgaagaaaatactttaaataaattgtaatgaattggttaagatgctgacttgaaatttctgattggctaatgctttaatttttattttttttgagacaagttctcactctgttacccagagtgagagtgcaggctggagtgcagtggcgccatcactgttcactgcagcttcgacctccccaggctcaggtggtcccctgcctcaacctcctgagtagctgggactacaggtgtgtgccaccacacctggccagtttttgtattttttgtagagacaggttgtcactatgttgctcaggctagtcttgaactcctgggctcaagtgatcttccagtctcccaaagtgctgggattacaggcctgagcagtcacacccagccctggctaatgctttgaagtcatgactcccattctgatttttaactagtcattactttcctgttcttccattatggtagtctcacaagacttataatttcagactttttattggtaactacaagagattattgttttagctttttcattttaaagatattgttttgtctagttgtattctgaaattacatgttaggtataattattagttgtacaattagctattaaattttcagaaagtttgaagggagagagtaagtgaaaagccactcctgctaaagattagacactgtactagatgcttggaggattaagaggagaccagagctaagggaaatctttgtctggattttattgacccattttggaattagctgcagatcttaagaaatcagattgggttggcaggacgtggtggctcatgtctgtaatctcagcactttgggaggctgaggcgggaggatcacgaggtcaagagattgagaccagcctggccaacatggtgaaaccccgtctctactaaaaatacaaaaattagccaggtgtggtggcgggcacctgtagtcccagctactcaggaggctgagacaggagaatggcatgaaccccggaggtggaggtttcagtgagccaagatcgtgccactgcactccagcctggtgacagagcgagactccgtctcaaaaaacaaacaaacaaaaaacagtttgggtcaataatttgcgtttacagtactagtttatgtacttatttgtcactttgtaatttttagggagttgcttttcaactgtaatctgtttttattaaactatttgaaatgagctttgtattcagtcactagttgtgtttctcccttccaagtactaaccaggctcaaccctgcttctgagatcagacaagatcaggagcgttcagggtggtttggttgtagacactaattgctttgcttataagccttaaggataccttacaattaataactttggtaaaacctcatttgaaaaataagactaagcctgagccaaacggcaaaatcccatctctacaaaaaattagccgagcatggtgttgggtgcctgtggtcccagctactcaggaggctgaggtgggaggatcacttgagctcgggaggttgaggctgcagtgagtcaagattgtgccactgcattccagcatgggtgacagagtgagaccctgtctcaaaaaaaagaaaagaaaaaagtaagactaaaatcagttagtgacaaaacttatgtattacagcatcttgacttaaaaaaaaaacaaaccaaaacccttcattttccgagtttttcataagccaggtattttcttaaatttgaagtcttagaaacttaggttattttggctcgctagctgtatgtgtagagtgaaaatagtatttctacatttggtcagttgcataatgttactatacctttgatccatctggaatttatcttggtatgcagtgtgaagcaagcatcggattctgcttttttctaggtgactacccagttgtcccatgacttgattttaaaaagcagctttattatataccaaatgtttatattttgggtatttttagggatttctactttgttccactaatctgtatttgtaccagtacgacacagctttcagtattgtatcttctaatattttaatatctgataggctagtctccattttcttcggagattttttcttttttttctgcgtttgcttaattgttatttcttttttttttttttttttttgagatggaattttgctcttgtcgcccaggctggagtgcaatggtgtgagctcagctcactgcaacctctgcctcccaggtttaagtgattctcctgcctcagcctctggagtgggttaggattacgggctcccgccacagctaatttttgtatttttagtagagatggggtttcaccatgttggccaggctggtctcaaactcctgaccttaggtgatctacccacctcagcctcccaaagtgctgggattacaggtgtgggtcaccacgcctggccgcttaattgttatttctacatgaactttagaatcagcttgtctaattaaaaaaaaaaaaaaagaaagaaaaacatggtacttcagttttcatttgcatcatggcaaatttgtatagcaacttagaattgatcttaataatgttgaatatcccgctcaagagtattgtatgtttttctaattgtctaagttttcttttgtgtctttcagtacaggtttagttttctccctttatgtcttcagtttattcctagttcttaactgctgtggtatattggtatgttgtgtgtttctcttatatttctttttatttatttcttttttttttttaaagagatgggatcttgcaatgttgcccaggctggactcgaactcctgggcttaagcagttctcttgtttcagcacattatatttattatctggccatgtatatatgaaggctgtttcagagtattaatttgtttcagaatattaattttgtatccagaaatagtttatcgtattctcttgtcagtttttgggttttcctgggaaagtttcttatctgagtttttatgctttttatttctcccatctaatatgaaataacaatgctaagcaattgtgataataatggatatctttgtcttgttgctgtccttcgtgggaatacttctgtagtctccccactagtcagcataatctggtatttggtattagttttttgttttttaaaactcatgtaaaggagatagccatatgatttttcttctttgagcattaatgctgaattatatcagcagaattgttaatgttggtgttggactgtatttatgcaaaaaatctcacttggacatggtttattcctaaaatgtactggtaattttggttgttacttttttttttaatcaaggattttcatatctgtatttatatattcaatttttccgaagttttttggcaccaatattatactcattttgtaaatacaaattagacacttttctatattttggatccattaaaatcccactggaggccgggcacagtggcttatgcctgtaatcccagcactttgggaggccgaggcaggcagatcacctgaggtcaggagttcgagaccatcctagccaccatggtgaaaccctgtctctactaaaaataaaaaaattagccgggcgcggtggctcacgcctgtaatcccagcactttgggaggccgaggcgggcagatcatgaggtcaggagattgagaccatcctggttaacacagtgaaatcccgtctctactaaaaatacaaaaaaattagccggacgtggtggcgggcgcctgtagtcccagctacttgggaggctgaggcaggtcaatggcgtgaacctgggaggcggagtttgcagtgagccgagatggcaccactgcactccagcctgggcgacagagtgagactctgtctcaaaaaaaaaaaaaaaaattagctgggtgtggtggtgggtacctgtaatcccagctactcgggaggctgaggcgggagaattgcttgagcctggatggtggaggctcaagcctggatggagtgagccgagatcaagctggttattaactcagcttcgtattttagttctattttttgaggtggagtctcactctgttgctcaggctggagtgcagtggcgtgatctcggctcaccgcaacctccgcttcctgggttcaagccattctcctgcctcagcctcccgtgtagctgggattacaggcatatgccatgagacctggctaatattttttgtatttttagtagagatggagtttcaccatgttggccaggctggtctcaaactcctgacctcaagtgatctgcctgccttggccgcctcccaaagtgctgggattacaggcgtgagccaccacgcctggcctgactcagctacttctgattttggaaatttatcatggttttctttgtgtcctaatatttacacaagttttgtaacatgttctttgggtattttaaaaagtgtgtcctgctttcaagatgcttttgatctaacaatataaattaaatctagattagagctaaattagatttgttatctatttctgcctagtttttgtttcctatcatttttgccttttgagtggtaatgctatgttatttggggcaattcagtattgatattcagtggtttgcactctttattgttaataaagtaaaacacaatggtttgcactctttattgttaataaagtaaaacacctgttacgtactccatgaagtctataacctagagtcatttgtatgcttactggtctagaaaccactgatttattctttgttcttttttctctctctgaagtcgttatctttcattagttacctatttttttttttgtttgtgttttttccagcgctctgttgaattacatattattgtatttccttttaatcctcttataggtgcttgttgcataccagtttttttcccccaattacaaagtatgtgtgtgttgatcatagataaatgtatgcatctatgctcagtacaggaaacctggaaaacagcacaaaaggaaaaaccaaaaggctaaatttgtgatcccaccattataaaaatatcttatccttatttgtttccatatccacataaacatacttctctcttttccacaatgtcagggcctattaaatcttgtttctatacgctagtgactccaattttcttttttaatttgcgataaaacgggtgcttaggaaaatctgaaccagtattttattccagctgaattaaaagaaattgatgcataaagaaaagcttactggagctattgaaatgttcctcagtatgaatcagggtactgtgacctcttgttggccttccattttcagccaagtacgccgatgtgcagcggacagtaataagggggctaagttttgcaaacacagagcttgtggacagtgatagatcctgaaagtagtgtctcttgctaattctcagacttaacctgactgaaagcaaggcctcacaaattgaattcagtcttcacagttacgttcttaactaacacaaaaaaatattctaatactattctgttcaaataaaaatttacgttttctggccaggtgcggtggctcacacctgtaatctcagcactttgggaggccaagatgggtggatcacctgaggtcaggagtttgagaccagcctggccaacatggcaaaaacccatctctactaaatacaaaaaaattagctgggcatggtggtgtgtgcctgtagtaccagctacctgggaggctgaggcaggaggattgcttgaacctgggaggcagagggttgcagtgagccgagattgtgtcaccacactccagcctgagtgacagagggagactccatctttaaaaaaaaaaaaaattttttttttttacatgttttctataggtatactagtttactggtagagtggttggaaatgttttgcctttttgaaagttatatttaattcaccaacctttttttttctttaaacacatcgttgctggtttgaataatttgtcaacctttgtggagctctttctgcatgtcaggcagtatataaaagtatcttttgttaatttttataataagaatcatttgagattactttatatgtggtataggggaatggtttccaggcttgggttttatggatcagtaagaaaaaaaatgggatgatgaacatatgattgctagcttttaattttgtcaatatagacattggtatcatttatcttgaatactcaatatttaatttggaaacatgaatatttttccttcatttattctctcattatagatttatggtctatctagagttggctggttattcagtctactacctttattttactgatgagggagctgaggctccaaaatgcctttgagaccccagagagaagacagaaaacttctttctcatgaatatattatagcctaatattgacatgttactagcagttacaaacataaatgttgctatgacttgtatgggagttaatttttcttcaggcttgaaatgtgatgattcccaaagccatctttttgtgtaaattagataatttcctggattaatttgtatcttgggaaattactgattgcagctattattttagaagtgtgactgatagcttactgagattctgagttgctattttgttttgggaagcatttttagagagtattataataatttgacagatatgcaaaaggaaaggaagtgaaaatgagagaacattaatacagttgcccttgggctttctcctccccttctcagagggcctggtccttcgcaaggtgttttttattattactttttaaaaatttcctggtctcctatccctgcctccttcaacttttccttttcccatttcttcccattccttaacccatctcttcttctttcattctggctcctccttctctccctgcatttctcttgttacctgtttccccatctctttttattcttagatttattctttgttcttttttctgatgtctttaaccagtaattcagattttaagttaccgggttttcttcctgtagcatttgaactgaagtgatactgagctggaaaaatagaatatcactgataagttgttggttttgggatttctaagttttattagtgtaaaagttcttgaattatgaatgtgtgttccaatccataccaccagctaaaaaagaaaaagaatatgttttattgttatttttgattgatactttccctttaatagaagcatatacttattttagtggaaatttatgattgattataaatttttttccctgtgattaattatagctttacattagtggcaaacatttctttcagtcttgttttgtgcacattttttacataaggcaatttaatttaatttattaaaacattttaaaatttataattattggccaggaatggtggcttatacctgtaatcctagcactttgggaggccaaggcaggaggatagcttgagttcagaagttcaaaaccagcctggttaacatagtgagacctcatttctattaaaacacacacacacacacacacacacacacacacacttatagttattttctatttattatatgctttttaaaaataaaataatttttttagttccatgtctactcctatgttatgtgcctttagtagacactcttagtgccatcacaacgttatcttctggattttccattatttaatcatttttccataattgagtatttagataataggagagtattgtaaaataaatgggtttagggaaaatacaaccctcctagcttaaatcaggaggaattggataccctgaacagaccaataacaagcagcaagattgaaatggtaatttaaaaattaccaacaaaaaagtccaggaccagatggcttcacagcagaattctaccagacattcaaagaagagttggtaccaatccttctgacactattccacaagataaagaaggaaccatgcctaattcattgtataaagccagcatcaccctaccaaaaccaggaaaggacataaccaaaaaagaaaactacagactaatatccttgatgaacatagatgctaaaatccttaacaaaatactagctaaccaaatctagcaacatatcaaaaagataatccaccatgatcaagtgggtttcataccagggatgcagggatggtttaacatatttaagtcaataaatgtgatacaccacataaacagaattaaaaacaaaagtcacatgatcatctcaatagatgcagaaaaagcatttgacaaaatccagcatccctttatgattagaactctcagcaaaatcagcatacgagggacatacctcaatgtaatgaaagccatctctgggccaggtgcagtggctcacatcacacctgtaatcccagcactttgggaggccgaggcaggtggattacctgaggtcaggagtttgagaccagcctggccaacatggtgaaaccccgtctcatctaaaaatacaaaaattagctcggcatggtggcaggcgcttgcaatcccagctactcaggagactgagacaggagaatcgcttgaacccaggaggtggaagttgcagtgagccgagatcgcaccattgcactccagcatgggcgacagagcgagactctgtctcaaaaacaaaaacaaaaaaacccaaccatctatgacacacctacagccaacctaatactgaatggggaaaagtggaaagcattacctgtgagaactagaacaagacaaggatgcctgttctcaccactcctcttcaacatagtactggaagtcctagccagaacattcagacaagggaaagaaagaaagggcatccaaactggtaaagaggaagtcaaactgtcactgtttgctgacaatatgatcgtttaccttgaaaaccctaaagcctcctccagaaagctcctagaactgataaaagaattcagcaaagtttctggatacaagattaatgtacacaaatcagaagctcttctatacaccaacagtgaccaagcagagaatcaaataaaaaactcaaccccttttacaacagctgcaaaaaaaaaaaatacttaggaatatacctaactaaggagtcaaaagacctctacaaggaaaactacaaaacactgctgaaagaaatcatagatggcacaaacaaatggaagcacatcccatgctcatggatgggtagaatcaatattgtgaaaatgaccatactgccaaaagcaatctacaaattcaatgtaatccccatcaaaataccaccatcattcttcacagaattagaaacaacatttctaaaattcatatggaacaaaaaaagaacctgcatagccaaagcaagactaagcaaagagaacaaatctggaggcatcacactacctgatttcaaactatactataaggccatagtcaccaaaacagcacggtactggtataaaaataggcacacagaccaatggaacagaatagagaacccagaaataaacccaaatacttacagccaattgatcttcgacaaagcaaacaaaaacataaagtggggaaaggataccctttttaacaatagcactgggataattggctggccatatgtaggacaatgaaactggatcctatacaagatagattaaggacttaacgacctgaagctataaaaaattctagaaggtatcattggaaaaacccttctagacattggcttaggcgaggatttcatgaccaagaacccaaaagcaaatgcaataaaaacaaagataaatagctgggacccaattaagctaaagagcttttgcacagtaaaaggaatagtcagcagagtaaacagacaacccacagagtgggagaaaatcttcataatgtatacatctgacaaaggactgatatccagaatctacaaccaactcagtaagaaaaaaacaatcccatcaaaaagtgggctaaggacatgaatagacagttctcaaaagaggatatacagatggccaacaaacatgaaaaatgcccaacatcactaatgatcagggaagtgcacatcaaaaccacaatgtgataccaccttactcctgcaagaatggccataataaaaaaataaaaaaacagtagatgttggcgtggatgcagtgatcaggcaacacatttacactgctgtgggaatgtaaactagtacagccactctggaaaacagtgtggagattccttaaagaactaagagtagaactaccatttgatccagcagtcccactactggatatctacccagaggaaaagaagtcatttgaaaaagatgcttgcacatgtatgtttatagcagcacaattcacaagtgtaaaatcatggaagcaacccaaatgcctatcaacgaatggataaactgctgtgtgtatatatgtatatatgatagtgtgtgtgtgtgtatatatatgtatataagtatgtatatatgtgtcatatatatatatatatgatggaatactgctcagctgtaaaaaggaatgaattagcatttgcagtgacttggatgagattggagactgttctaagtaaagtaactcaggaatggaaaaccaaacatctaatgttctcactgataggtgaaagctaagctatgaggacgcaaaggcataagaatgatacagtggactttggtgacctggggggaagggttggaggggggtgagggataaaagaccacaaatagggtgcagcgtatactgctcgggtgatgggtgcaccgaaatctcacaaatcaccactaaggaacttatgtaactgaataccacctgtaccccaataacttatggaaaaataaataataaaaaaagggtttaaaaaagtataggaagttattggtgaaacgtgtgtacatatatatatgtacacacacacacacacatacatgtatgtacatatatataaaagatgttctgggggaagaaaactgaccctaaataggtattaaaatgcctttgagctttgagagactgaagttttgctgcatagtggaccttagaaaaatctttatgtgcccaaatatagatttctcatattcttaaggactgtggaaaataatatataatatgggaaaaaaatattttatcagctctattgaggcatgatttaaaaaattttacattatttactaataaagagaactgtattttatttttcatttactattccttgtggtcttctatctagttgttcactgcctcattgcctctctgattttcaaacttggaagttattgacacgcgtctttctggccctctcctctggagcacctctctgctgccaatttttttctgccatttttttttttgtaatttaatttaaaaaaaaacttactgaggtgaaattcacatagcatgaaattaaccatgttaaaatgatcaatttagtggcatttagtactctcatagtgttgtgtaatcaccatatctggttctgaaacctttccttcactccaaaggagacccttaccaactacacagtttctctttcccagacccaggcaaccgccagtctgctttcagtctctttggattttccctttctggacacctcatatgaatggcatcacacaatatttgtgaacttttatgtcttcttttttttattttaaattttttttagagctggggtctcactctgttgcccaggctgcagtgcagtggtgcgatcatagctcactgcagccttttaccacctggactcaagtgaacctgattagctgggactacaggcatgcaccaccacaccatgctaattttttttttttttttttttttttttttgagactggctctcttgcccaagctggagtgcagtggcttaatcatggctcacggcaacctttgcctcctgggctcaagccatcctcccacctcagcctcccaagtagctgggactacaggtgcgtaccaccatgctcggctaatgtttgtatgtttagtagagatggggtttggccatattacccagggtggtctcaaactcctgagctctagttaattttgaaatatagaatatattattgttaactgtagtcaccttactgtacaatagaataccagaacttattcttcctatgtgatggtaactttaatcattcatcaacttctccccatcccactccctgcccttccctttactaccctccccacctctccggcctctggtaattactattatactctctactcttttgggaagggtgtttgctatttatttatgataaatattccacatctgtgattctccacagtcagaagttctttgagaggatgccatcagccttggtcagtcagagaatggagtcatctgatttaccatcctgtgaatggccctaggcagatagcataggtgattgttgttactgttttgagacagaatctctgttgcccaagctggagtgcagtggcatgatctcagctcactgcagcctctgcctcctgggttcaagtgattcttctgcctcagcctcccaagtagttgagactacaggcatgtgccaccatgcccggctaatttttgtatttttgtagatactgggtttcaccatgttggccaggctggtctcgaattcctgacctcaggtgatctgccttcttcggcttctcaaagttctaagattacagtcgtgagccaccgtgcctggctggtggcggtggtggtttttttaatttttttaatttttgttttttcaacttttattttgattcgggggcacatgtgcggtttgttacctgagtaacaaaaaaaaaagcctctgcctttatgagataaacttttttatattctgtatgagtaagatcatacaaaatttgtctttctgtgtctggcttgtttcagttaacacaaagtcctgcagattcattcacattgtcataaatgacaggatttcgttcttttttttttttttttttttttttttttttgagacagacagtcttgctgagttgcccaggctggagtgcagtggcaccatctcggctcactgcaacctccgtctcccagcctcaagtgattctcatgctggatttcattcatttttttatggtgaacagttgagccttttaaaaaagccaagcatctggctgggtgcagtagctcgcgtctgtgatcccagcactttgggaggccgaggcgggtggatcacaaggtcaagagatccagattatcctggccaacatggtgaaaccccgtctctactaaaaatacaaaaaattagccaggcgtggtggcgggggcctgtagtcccagctactccggaggctgaggcaggagaatggcttgaacccgggaggcggtggttgcagtgagccgagatcatgccactgcactccagcctggtgacagagcgagactccatctcaaaaaaaaaaaaaaaaaaaaaaaaaaaaaaaaaaaaaaaaaaaagccaagcacatccatttaaattttggtggaacttctttataacattcctggtgtcagcatattctaggtgttgtttggaaaattatttattaaatcaaagactcagcctctctcaagttctttacctcaacaacagttgcttcttcaaacagctgatatcacacaatttttattgtggttatagaaatgaaacactaattttctgcatatgtctgtgagcatattcacaactcattgctggaagtttagataccgctactttcccaatttaggaaaacagatttctggtatgtgcctacgtgggaaggttgaggctgctaagagtcttgcttctgggttttagaagtctcactgtgagaccgggtgcagtggctcatgcctgtaattgcagcactttgggaggctggggtgagtggatcacttgagcccaggagtttaagaccagcctatccaacatggcaaaaccccgtttctactaaaaatacaaaataattagccgggtgtgatggcacactcctgtaatcccagctactcagggaagctgaggcatgagaattgtttgaacctgggaggcagaggttgcaataaagattgtgccactgcactccagcctgggtgacagcaagatgcctgtgaatgttttctgcacatataaaaaggttaaatcattttcagtatggtttgctggttctttgaaaattgataatacatacactttataaatttaattctccatgaactacaatatcaaacagaatacactctatctctacaccatgctgaaaaatggaaactttttttttttctttttggtaacaggatcttgctgtgtcgcctaggctggagtgcagtggcacaatcactggtcactgcagccttgacttcctgggctcaggtgattctcctacctcagcctcccgagtagcagggactacaggcatgcaacaccacaccggcaaattttttgtatctttgtagagatgaggtttcaccatgttgcccaggctggtcttcgaactcctgggctcaagtaatccacctgcctcagccttccaaaatgtgggattacatgtgtaagtcaccacgcctggctgaaaacagaaacttttcaagaaaagttttaaaagaaactaccaaattaatagatgttaccatataacgttcagaaatacaaatattttatgaattatttccagtcctattacttggaggcaaccattgtaaatattttttatgtacaagttgagtatccctcatctaaatccttggcaccagaagtgtttcagatttcagattttctaactttttgcctcatacttattggttcagcatccctaatctgaaaaaaatgcccctgtgaacattgagttttgtcttgtctccctcccctcccctcctttcctctcctctcctctcccctcccttcccctcccctccccctcccccttcccctcccctcccctcccctctcccctcccttctcctctcccctttcccatctccactttcccctctcccctcccccttccctctcccttccccttcccctcccccttccctctcccttcccctcccccttccctctcccttcccctcccctctcccttcccacctcccccctctcccctcccctcccctcccttctcccctctcccctctcccctcccctcccccttccctctcccttcccctctgctcccctcccacctccccctacccctccccctcccctcctctcccctccccttcccctcccctccccctcccctctcccctctcccctccccttccctccccctcccctcccctcttccctccccctcccctcccctcccctcccctcccctttcctctcctgccctctcctctcttttcttagacagggtcttgtgctgttgcacaggctagagttcagtggcaccttcttaactcactgcagcctcaaatcctgagcttaagtgatcatctcacatcagcctcccaagtagctaggacaataggcatgcgtcaccacgcccagcgaatttttgattttcattaattaattatttttgtgtgtgagacagggtctcgctctgttgcccaggctgtagtgcagtggctcactgcaacctcaatctcccgggcacaagcgatcctcccatctcagcctcccaagtagctgggactacaggtgtatgccaacacacccagctaattttttcaattttttatagggacagggtctccctatgttgcccaggctggtctcgaactcctgtgctcaagtgatcctcccagctcggcctcacaaagtactgggatataggagtgagccaccaagcctggcctcggctaatttttaatttttttaatatagagatggggtctcactatgttgctggtctcaaattcttgagctcaagtgatcctcccacctctgccgcccaaagcactgggattacaggcatgagccacagtgcctggatcttttttttggactttgggagtgagacgtgaggattctgtcagccctaaaaccttaggtcttctggtttcttttttgtactctcaattttcttcgtactaccttcaaggtataatgtgcctaagaaatatttccttgtttttcaggttctaaatggcttctaagaagttgggtgcagattttcatggtaagtggactattagaatcatggtttaagttatgatttgatcagaggatttggaatcacttagcatagttttaagtacaagtgcccctttttgtctcttttaatgaacagcttgacaaaaataaagttgtacaacaatgtatcaagtatatctgaaaggtcttcagagaacttgaagtatttcttcaaatgggtagttaaactttagagtgtctctggttattgtacttgaaactactagtgatagattaggagtctgcctgttctggttcttcagcagcattgaaggtatagttactggctgggcgcggtggttcaagcctgtaatcccaacactttgggaggctgaggcgggtggatcacgaggtcaggagatcgagaccatcctgtctaacacggtgaaaccccgtctctactaaaaatacaaaaaattagccgggcgtgttggcgggcgcctgtagtcctagctactcgggaggctgagggcagaagaatggcgtgaacccgggaggcggagcttgcagtgagccgagatcgcaccactgcactccagcctgggggacagagcgagactccattaaaaaaaaaaaaaaaaaggtatagttacctttccctttcctgttccttggtcatagctcttctccatatgtgtgtgtgtgaaaaattctaaccctttggttaatgctttataatgttttagtttagttccctgtgccccatcaaaggaagtaatttactacaaatcaatcactatattacagaggtatcaatactgatatttataattttgaatttgaaattaagagtttctctgtcatcttttcctaagatacaaccttccaaaatggaatacaataataaactactatctgcctttggaggggaatggagggaggtggaggtagctaagaagttaaaattatacccttttttcatacagtagaaggtaagaatttaaaaccagaagtagccaagtaacacaattattttaagagaggttttaaagccaatcttccatgttaaaaattatatcagccaggcacagtggcatgtgcctgcagtcccagctactcagggggctaaggtgggaggattgcttgatcccagaagtttgaagttgcagtgatcatgccactgcactccagcctgggtgacagagtgagaccctgactcaaacaaaaaaaatacatatatatatcacatcattttaagaatgatttactagactgcctggggacgtgttggtatgctcttctgagctgccagtggttgcttgataagatcactcagctgagtcagagtggtgtagcggtgattgtgattttacttgcttatatattttacacaacagcctttgaattttgatttgtcagtgtgcatatacttacatagataacctgcctgagtttttaaaaatccttttgaagttattcaaactctggaattagcattccttaaagaggtcaggcatcttatttttcaggtgagctatttcctattatttatggattattacaggtcttcttaaaagtattcaaatgatagtagaaaggcagatctgggcagggcacggtggctcatgcctgtaatcccaacagtagattaggaggttgaggtgagaggatcgcttgaggccaggagttcaagaccagcctcagcaacatagtgagacgctgtctctacaaaaaaattttaaaaattagctgggtatgctggtatgtgcctgtagtctcagctactcgggaggctgagaggtgggagtagtgctgagcccgggaggtcaaggctgcagtgagccatgatcacgccactgcactccagccagaatcacatgagagcctgtctcaaacaaacaaacaaaaaatgattcttgccactgagcttaagaaaagaaaaagggaaaaaaaggcagatctgaattccctctagatcctaccttttcaagggagaaaagagaggacagagccaagggcagaggaaaagcttagggagagaaaatagcaaaaatgaaaaatttacacttatttcaaaagatagactttctgttttgaatctttggaacatctgttttgatcagactgaaaatagttggaccacatgttttgtgtttcaactgaacattccagagagaagattataattctgaaggtgtctgttcataaagactggtatttcccatatctccttaaattcctaaccaggctgggcatggtggctcacgcctgtaatcccagcactttgggaggccgaggcgggcggatcacctgaggtcaggagttcaagaccagcctgaccaacatggagaaaccctgtctctattaaaaatacaaaattagccgggtgtagtgacgcatgcctgtaatcccagctactcgggaggctgaggaaggagaatcgcttgaacctgggaggcagaggttgcagtgaactgagatggtgccaatgcactccattccatctcaaaaaaaaaaaaaaaagaattcttaaccaaagtgttattttgtttgtttgtttgtttgtttttgagacagagtctcgctctgtcgcccaggctggagtgcagtagcacgatcttggctcactgcaagctctgcctcctgggctcacgccattctctgccttagcctcccgagtagctgggactacaggtgcctgccaccatgcccagctaatttttttgtattttttagtagaggcggggttttaccgtattagccaggatggtctcgatctcctgacctcgtgatccgcctgccttggcctcccaaagtgttggggttacaggcgtgagccaccgtgcccggcccaaagtgttattgtttaaatgaaactttctacctgagtaaaatttaggagaaaaattttctttctcccttaggctgcttctcctttaacagggctctttttgctgagttcctggttgaatttcactggctttacttgttttttttgtttttgtttttttgttttttgggttttttttgggaacaaaacaataaacaccttcattacatgggtgaagacaaaacgaggatttatttgcctttccggaccttgattttcctaagatagaactccaactccttcccctctagcacatagccgtctgtttggccacactgtcccggccttgaagtgatgcacgcaagaagcttgccctgctggaactgctcctccaggagactgctgattttggcctttttttttcctttcatcgatttcttctgaaattttttagatcattttttgtttaaaatctcttcttcctcaggagtcagcttgtctcctttcttgtggcccaggggtagcgcatagtgggactcgtaccactgtccatacggtgtgctgtcgatgagtatggtgtgctgtcaatgagcatgaagcaattcttcaccagggtcttgttatggaccagctcgttattaaacgcattgtagacaacatcgatgatccttgttttgcaaatacaacgctgagccccaggagaaattccctacatgcagccttagggcacggtatttcttgttacctcctcaaacatggactgtgtggacgtggcaggggccaatgttggtgttggcagctgggcacccctgctcatacttccgcttcttgtggtagggctttctcttgcccccggtcttgcggtgcttgtgtcagttctcccgagagatgcccattgctcggcgctggctggaaagagctagttgggtttttagagtaaagtatcctatttttagatatcaggtagtttaaataatagaaatgaaaggcagtagatggagaatatttaataggcaattgtgcaggcatgtgagtacagaaaataaagtttctgctctctcaaggctaaaattagaaagccaggaactaaggctactccttccacttctcaggaagctgcttagcctttcttcactatatgtctctgtgtctgtgatccattattattcagttcatagacttgctttcttggcctaatagtgtagagtacacagggctataaatggccaccccagttaaggtgctgcttgctacttgatctttcagtttcagtggctagcatcatagagatagagattttatatcttttggtcaaggcaaactgatggcatagccttcttagcttatgagcctgtggattggtcctccaaagttagatatctgactgatccattgagaggttaatggatgtgggtaacttttctgtggcacttggggtgtggacaaggaggtaataactgctgtcatgacggatattaaaatgtatttttttttctttttaatatttgggactgactaggtctggtgctgtgctgtctttgactagcacttgatttacctctgagttcattattttcaactaaagttgaacaacttcactgttactagtgaccatatatccctaaataacagaataagggaaacaaacagttatttctcaaacttctagaaatattatccatttcttataaaaactccatacccttcgcttagtttggataaggactttcttggagcctggtgctttaaaatctcaatcctctggctgggcgcggtggctcatgcctgtaatcacagcactttgggaggttgaggcaggcggatcatgaggtcaggagatccagaccatcctggctaacatggtgaaaccctgtctctactaaaaatacaaaaaaaaattagccgagcatggtggctggtgcctgtagtcccagctacttgggaggctgaggcaggagaatggcgtgaacccaggaggaagagcttgcagtgagccgagatcacaccactgcactccagcctgggtgacagagcaagacgccatcttaaaaaaaaaaaaaaaagaaaagaaaaaagaaatctcaatcctctttcactgattcttattttcatctaggagaaaggttatttatctggaaaatgtgttgagactctaatttatgaaaaaatagaccccgtgataagatttgataaaactagcccccctccttaaatcactttatcgaggtataattgatatataaaaagctgtacatatttacatgtttaatgtatacatcttgatgagtttgaggataagtatacactcatgaaaccatcaccatcattaaggcataaacattcattaccttctaaagtttcctcctactctattgtgtgtgtgtgtgtgtgtgtgtgtgtgtgtgtgtgtgtgtgtgtgtgtgtggtgggctggagggattgcttgaggccaggagtttgagaccagcctgggcaacatagaaaaaccccgtttcttgtttttttttttttttttgagatggagtcccgctctgtcgcccatgctggagtgcagtggtgccatctctgctcactgcaacctctgcctcctcaagcaattgctcaagcaattctcctgtctcagccccctgagtagctgggattataggtgcgcaccaccatgcctggctaatttttgtatttttagtagagacagggtttcgccatgttggccaggctggttttgaactcttgacctcaggtgatccacctgcgtctgcgtcccaaagttctgggattacaggcatgagccaccgcgcctggccttgtttctacaaaaaattaaaaattagccaggtgtggtggagcttctctatagtcctagctgctcaggagcctgggacaggaggattgcttaagcctgggagttcaaggttatagtgagctatgatcacatatgcattaggttgtttggtattgttccagatatctcttaacactgttcaattttttttttccctttttctctctttgtatttcagtttggctaatttcttttgacctaacttcaagtcccaagtctttcttatgctgattattgtcttcggattagcatagcctggtttatgaaccatcaaaatttttcttttctgataatgcatttttcacttgtggcatttccatttgtctctttatatttccatctctctgctgaagttcccatctgctcacacatgtttagtttttccattagaccctttaacatatttattattgttattttaaagctccctgtctgatgtttacaatatcttggctctccctgaatttgggtctgatgactggttcctctcttaattctggatcacattttcttgcttctttctgtgtcttataatttttaattgaatgccagacaatgtatattaaaaagctgattttgtagattatatggcttgttttgttgttaggatatgagtaacattttcttgtgatttcacatcttacaacagatatctatgttattttgtatatttatgattttgccagtgttacctggcttgtggctttaagatagttttttttgttttatttatttatttatttatttttaagtcctttgctgcaggaggcgctgatggctttaagatggtgataaccagtcatggaattacagattttcacttaattttaagtttgatcttctttttaaaaaaacctcaatttgaatctcctcacaataatacctgaattaaatattatgcatttgctatcctctattggggatttctccccccacctggaattgtactttttttagaaaaaacgggcagattgcagaatcataggcatatcagcaatttagaaagaaacaaagcaaatgaagtcttcccagagtttatcctgagtcattacatattcagcccactcactatcttccaccaaagtggtatgcagaacctaacttcaaggaactgccacaaccgcagccatgaggcatgcacttagtggctatttaaaaaattgaacttaggaaaatgggtgcaaatattttaggaagattttcagtaataaggactgaaactaaaaggtatttgaggttgaggcagaaaattgttagctataatattttaaattctagatacattggataaaaatggacaattccactttgggaggctgaggtgggaggattgcttgagtctgggaatttgagaccagcctgggcaacaaagtgagccctcagctctacaaaaaaatttaaaaaattagctgggcatggtggtgcatgcctgtagtctcagctactcaggaggctgaggtgggaggatcacttgagcccaggagaatgaggctgcagtgagtggtgatgacgtcactgcactccagcctgggtgagagagcaagaccctgtcttaaaaaagaagctgggtgcgatggctcacgcctttaatcccagcactttgggaggccgaggtgggcggatcatgaggtaaggagatcgagaccatcctggctaacacggtgaaaccccatctctactaaaaatacaaaacattagccaggcgtggtggcaggcacctgtagtcccagctactcaggaggctgaggcaggagaatagcgtgaacccaggaggcggagcttgcagtgagccgagattgtgccactgcactccagcctgggcgacagtgcgagacttcatctcaaaaaaaaaaaaaaaaagaaaaagaaaagctaattcctgaggcataattggttatatttggaagacatatattttgtaattctcacagcaccatcattaatagtacttacggtggtctctatccattattacaaatactgctttctttgtttcccccccccccttttttttttggagatggagtctcagtcacccaggctggagttcagtggcgtgattttcgctcactgccgcctccgcctcctgggttcaagtgatcctcctgcctcagtttcccgagtagctgggattacaggcgtgcatcaccatgcctagctaatttttttttttttttgtatttttagtagagacgggggtttcaccatgttggccaggctggtcttgaaatcctgaccttgagtgatctatccgccttggcctcccaaagtgctgggattacagctgtgagccactgtgcctggccacgttcccatctctttaaatgcctttttgccctttatccctacctcctccttccagtaatcactgaactatttgttcattctctcttaataaagcttttaaagtacgaagaccctggagggttgggggatgtactaaataaatcaccttggcattggctttattagtgaaaggaaactgatttttaaaccagatacctgcaattggtgctagtgaagggaaacggcagccctttaaaggtattctacctcatcttttctgtccctaagccaggacctagtttcatttatagaatataagtttgtcagtgtatatcacactgtagtagtttgctacgtgaaaggtaggcaaggagatcacttctcttataggatttttatctttttttttttttttgagatggagtcttgttctgttgcccaggctgcagtgcaatgacgtgatcttggctcactgcaacctctgcatcccaggttcaagcgattctcctgcctcagcctcccgagtagctgggattacaggcacacaccaccatgctcggctaatttttgtatttttggtagagatggggtttcaccatgttggccaggatggtcttgatctcctgacctcgtgatcctcccacctcagcctcccaaagtgctgggattacaggcatgagccactgtactcggcctgacatggagtcttgctctgtcactaggctggagtgcagtggagccatctcggctcactgcagcctctgactccctggttcaagagattctcctgcctcagcctcctgagtagctgggattacaggaacgtgctaccacgcccagctaattttttgtatttttagtagagatggggtttcaccatgttggccaggatggtctcaatctcctgacctcatgatctgcttgcctcggcctcccaaaatgctgggattacaggcataagtcaccatgcttggcccctaatttttgtatttttagtagatacggggttttaccatgttggccaggctggtctcgaactcctgacctcaggtgatccacccgcctctgcctcccaaagtgttgggattacaggcgtgagccaccacgctggcctcatatctttagcacagtacttttgtagtttttctccaactccttgttaccaatctacaatagaacttggttatttaagaaaatttagttgttattagtattattattgaaatggagtctcgccctgtcgcccaggctggagtgcagtggtgtgatctcagctcactgcaacttctgcctcccgggttaaagcaattctcctgcttcagcttcccgagtagctgggattacatgggcacgccaccatgcccagctaatttttttgtatttttagtagagacggggtttcaccatgctggccaggctggtctcgaactcctgacctcatgatccgcctgccttggcctctcaaattgctgggattacaggcgtgagctgccgcacctggccaaatatagttattattaatgtggatagattttatcatttttgtgtatcatctgtgcttattagtgttaaacaaaagacttaattgtgtgcttgttaacttgactgtaaatctttccaatgattcttagtgccaaagtttcagctttgtttccagttaatcaggatactagccaaagttagaatctttcaataacatttgagtccctattttcaaagaaaacagacaaaaattctttaaaattagaaagagcttttgttctaatcttttaaaatataagcattttataagtgccatagagtttaagtgttgtaatcaacacttagttttgttggtaagctgtgatggcacccggcacttttttggtggtgctggggcgggggatgacatctgtctggtacatttttgtaactttgttcagatctcagacatatgcttgtatcatgtatgagtaatctgttaaaaacattttcatgactagccttgtcatttttgggcaaataattttcaattttcatattaaatataagttggaactgggtgtgatagctcatgcttgtaatcccagcaatttgagaggccgaggcgggaagaatgcttgagcccaggagttggagaccagcctggacgacataatgagaccctgtcttagctactcaggaggctgaggtgggaggattatttgggacccagagggcaaggatgcagtgagccatgatagcaccactgcactccagcctgggcaaatgtctctttaaaaaaattatgtgtgtgtgtgtgtgtgtgtgtgtgtgtgtgtgtgtgtgtgtgtgtgttggggtcggggttagattgaatccagaagaagtataaaagcatgtctttatttatttatttattttgagatggagtttcactcttgttgcccaggctggagtgcactggtgcgttcttggctcactgcaacttctgcctcccaggttcctgcctcagcctcccgagtagctgggattacaggcatgtgccaccatgtctggctaatttcttgtatttttagtagagatggggtttcaccattgttggccaggctggtctcgaactcatgacctccacctgccttggcctcccaaagttctgggattacaggcgtgagccaccggtcccggccaaaagcatgtctttattattggttcttcattcattgaacaaattccatgagccatacattatactgagttttggtgatatagatatgaaacatttctcccctgttaagggacactcaaaatttttttgttttttttttttgtttgtttgtttttgagatagagtctcgctctgtcacccagcctggagtgcagtggtgcgatcttgactcactgcaagctccgcctcccgggttcacgacattcttctgcctcagcctccccagtagctgggactacaggcgcccccactgcgcccggctcattttttgtatttttagtagagacggggtttcaccatggtcttgatctcctgacctcatgatccgcccacctcagcctcccaaagtgctgggattacaggcgtgagccactgtgcccggccgggacactcaaattcttgctcagactctctgaagggaggttaaggaaatgaactattaacaatacaaagaggtaagtcctaaggtggtagtattaactggctattctgaggttgagaaaggagtcgttcttgaggctcactgcaccttccgcctcctaggttcaagcagttctcctgcttcagcctcccaagtagctgggactacaggcgtgtgccactacgcccggtgcagttttgtattttcagtagagagggggtttcaccatgttggccaggttagtctagaactcctgacctcagatgatccacctgccttggcctcccaaaggggtgagactacaggcatgggccactgcacccggcctgagtttcttcttaaagggctaaataataattactcaagtaaataagggtaggaatgtgataggcattgccagccatgaaattagcacatctaaaaggatgaccacacttggaaaattcctagggtgttaataaatatggtgatttcctagagtgttaaattactaaattgatcacagttgtaaatttaatttgctagctaagtgagcgatagttcaaatgttttagttctggccagaagttagatgggaaagagatggaaaagacacccattcccccaagggttgggagtagggggcattcttcctaatggagagtttcaggtgtatctgcagaatacctatcccaggaaatgggagtgatgacaagattacaaatattttctgaccttgataattcttgagatctacacagttttcgtaaggaaagaagtcatcttcatggaagaggtataattgtcttgttctgtgtctgtgagcacccaagaaaatagtgtgaggctgagaggtaaaccttcctttaccagctgccaatgttgaaaaacaaaatttattttatcaactgtatttcattgtccacaatggttctgcctgtaaattagcctcccagagctgctccagaaaagaaaacatggccctgtttggagtatatagctatcgagagtctcatataagaggcgttttccttttatctgaccccgtgaatagatggtcatgttctggtctttccatgttgttcagttatgatgggcaaagaacctgtaggagattaaagcagctccattgtgtatggcttgtgctcctgatgatcttactcagaatcccatgattttgtcgggttttccccttttcatgtaagtgttgtgctgcttaaggatttccatgttgttctagaatgtagtgttttttaagctagagtaattctttctcttcttttttttttttttgagacagagtcttgctctgttgcccaggctggagtgcagtggcgcgatctcggctgactgcaacctccgtctcctgggttcaagcgattctcctgccttagcctcctgagtagctgggtttacaggcatgcaccaccatgcctggccagtttttgtgtttttagtagagacggggtttcaccatgttggtcaggctgggagtaattctttatattaattgaaaatgatttttttctaaagtagagaatcctaatcaaggagattcttttctggggagtctcatccattcagtatttattaaatgctgatgtgtgccaggcaatactgaaggttttgaaaatatgtcattgaataagatagacttaaatacttgccctatatttctttgggaaagagaaacaataaatataattaaaaagtcaatttataatgttagaaggcaataagtgttatggggaaaaggtaaatggtaatatgagagtattgtagtgagggtggggtagtacttttaagtagggcgggtcagggtaagcctcactgagagggtaacatttgagcaaaaacttgaaggagtgagtcatgtgacagaagagtgttccagttagaaggaacagttggtacagagtatttgaggtggattaagcctgtcatctttaaaagcagcagtcaccattacttagtacagtgagccagaggatgactgtaaggagatgagggcagagaggctgtgggtgatgtgagtactttggcttttacactaagtgccattgaaaataaactcctaataagactgaatcagtagaatttccttctcagagatttagggtcaaggaaaaggtaagcagtcagagaaggaactctgtaacttctagacttaggatagcaaaaattaatgcatatccgttagacataagattatggcaaaacaaataatgaagttctttgctgatattttctactttatttttgattatagggactttcagttaccttgatgatgtcccatttaagacaggagacaaattcaaaacaccagctaaagttggtctacctattggcttctccttgcctgattgtttgcaggttgtcagagaagtacaggtaagtggtaatttttagttaaagtttagtgcatttaaaagtttaaagagtaagaatttgatgtatagtgaaatagaaaaaattacagttgtgagctgaatcatgttttggtcaacgatggaccacatatacaatggtggtcccataaaattataaggggctgggcgtggtggctcacgcctgtaatcctaacactgtgggtggccaaggtgggaggatcacttgagctcaggagtttgagaccaggctgggcaacttaatgagacctcatctctactaggggaaaaaaaaatgatagatatatatatatatattcctatcacatagtgacgtagtagccattgtaacatggtagcgcaatgcattactcaagtgtttgtggtgatgctggtgtaaacacatctgctgcattgacagtcttacaaaagtatagcacatacaattcagtatagtatgtaatacaaataaatgactatgttactcatttatgtattactatgctatactttttagcattatctttctacttattaaaaaagttaactgtagaacagcctcaggtaggtccttcaggagatattccacaggaaggcattgtgatcataggagatgacagctccatgcatgtattgctcctgaagaccatacagtgaaacaagatgggaaggtggaagacagtgatactgatgatcctataggcctaggctgatgtgtgtgtggcttcatttttaagaaagaagtttaaaaagtaaaaaaataaaaaatttaaagatcaaaaagcttatagaataagagtataaagaaaaatgttttggaagataaaaaacattttaggctaagtattagttcggaagagtaaaaaagttaaaaaaatttaatgtttataaagttaaaaaggcacagtaagttagagttattatttagagaaaaattgttttttacatttgtgtatatgtgtatatatatatgtgtgtgtgtgtgtgtgtgtgtgtatatatatatgtatgtatttttttgagacggagtctcactctgtcgcccaggctggagtgcagcggtgcaatcttggctcactgcaagctctgcctcctgggttcacaccattctcctgcctcagcctcccgagtagctgggactacaggcgcccgccaccatgcctggctaattttttgtattttttagtagagacggggtttcactgtattagccaggatggtctcaatctcctgacctcgtgatctgcctgccttggcctcccaaagtgctggaattacaggcgtgagccactgcgcccggtcaaaaaattgttttttacaaatttagtatagcctaagtgtacagtgtttataaagtcaagggtactactgtacagtaatgtcctaggccttcacatttaccattcgcttactcactcacccagagcaacttccagtcctacaagctccatttatggtaagtgccctatacaggtggaccatttttaatctttttttttttttttctttttttgagacggagtttcattcttgttgcccaggctggagtgcaatggcgcgatctcgtcaagaagagagactctttctgtctctatgtaacctccgcctccacctcctgggttcaagcaattctcctgcctcagccttctaagtagctgggattacaggtgtgcactaccatgcccggctaattttgtatttttagtggagtcagggtttcaccatgttagtcaggctggtctcgagctcctgacttcagttgatccgcccacctcgacctcccaaaatgctgggattacaagcgtgagccactgcacccagcctatttttaatcttttataccatatttttactgtaccttttctatgtttagataggtttagatacataaatacttgccattatgctacaattgcctacagtattcagcatagtaacatgctatacaggtttatagcctaggagcaatactgtaccatagcaataccgtatagcctaggtgtatagcaggctgtaccatctaggtttgtgtaaatacactctgccctcaccgatgaaatcacctaacatgcattttttagagcgtatccttgtcattatgactgtattatcttatttttgtttcttatagctcctattgttgcctggctcacattgttatacacacatacagattttgtgtcctctgttagattgtatattcctggggccctgtacctcacacagaccaaattcccattgcccatttaactagctttttcacctctttccctttctctgctaatgtcctcattctcttttcagtcactcaagttagatttgctgtgtctcacattgcagttcagcctgacctcatttttcacctgggctgttgcagtagcctctttaattggcccttctgcttgcggtgttttccttctattttttacatagtttctagaatgatcttaaagttttaaattgtcacatcccttttcataactgttcaaaagggttcaatgactaaccagtccttattgagtatagtctagacataaggttatgacaaagcaattgagctcatcacctcctccatgttccctccaaaccacgttactagtagtctgttagaccttgtacgttatctctctatatgttgcttctgctcatgagccgttatctccacctggaatattctttttctcactatactgaaaactccgttcaaatctattctagattgcgtgtaaatgctatctcttcgagggcttctttctcagtcctcccatctttaaggctcagttatatagaaatgtactgagttactccttgaaagcaacaaatagccctgtgttacttacttactgcagtacacatttactaaacgcatgtttatgagaggtggtctactgggcactgcaggtagagataaatgccttaatcactgccctcaaggtacttacagttgagagaagttacctagagcaacagaaaaatatggtactttaactcttcttcctgtgacttaagaagatttttaaaaatgcagctttcaggaaaagaagttttgtttttctggaattcactaaggcatcaccattttattctaaaaggtattactttttttatcttccaaagtctatatgcagtaagtaatcataatgagggaacttcagttacttttgggagaaaaaaaatatttattgagatataattcacataccatgaaatttacctttttttaaaaaaaaactttttttggtttatttgtagacacagtctcactctgtcactcagggtggagtctagtggcacagtcatggctcactgcagcctcgacctccctgagcttggtgatcctccctcctcagcctcctgagtagctgggactattggcatgcaccaccatgcctggctaatttttgtattttttgtagagatggggtttcatcatgttgcccaggctggtcttgagctcctggcctcaagtgatctgcccacctcagcctcccaaagtgctgggattacaggtgtcagccaccatgcccggccaaaattcacctttttaaggtatacatacagttcagtgatttttaatatattcacatagttgtgtaataatcattactatctaattcccgaacattttcatcaccccaaaaggaaacccagtgtccactagaagtcactccccacagcctctggcagcctcctcgcagcctctggcaaccagtatctactttctgtctctatgggtttgcctattctggccatttcatataaatggaattctgcagtagcagccttggtgactggcttattcacttagcataatgttttcaaggtttatccacgttgtatgaatgaggtattagtacttcatttatttttgtaggtgaataatatttcattgtttggatataacacactttgtttatctgttcatcagttgatgggacattgtggtagtttctaattttcggctattatgaataatgatgctctgaacatctgtaaacagatttttgtgtggacacatgttttcagttttcttgggtataatgtataggaatggcattgctgggtcatagtaactctatgtttaacattttaaagaactgctatttttccaaagtggctgcacaattttacaatcccagcagcactacaagagggtaccaatttcttcacttccttgcccactcttattaactcgttttcattatatacattcttgtgggtgagaagttgtctctcattgtggtttttatttgcatttctttcatgtctaatggtgtcaagcatcttaagtatctttaatactgaattaatgttccttttgtgtatggagactttattgggccagaagagagatagacctactttgtaacgtttttccatgttttatttgtgtaagaatttttttattggcaataacaaatagtcattacttacctttgtacatgatactattagcaatatgatttaaaaacacctgtcttgtttctcattattcactgctttactgatgccttcagaaaacttgttttgattcccaaattgtcgatttaggctttgctgggaatttcccatttggtcagtctgaagcaccaataaaagcatggtatgtgctcactgtgctcatagcacccactgataacaaaaggagatcaaagctaagagctccattccaatgtgggccatgtgaaatatttttgttctctaatgcacaaagcaatctttttttgagatggagtcttgctcttttgcccaggctggagtgcagtggcgccatcttggctcactgcaacctccacctcccaggttcaagagattctcctgccttagcctcctgagtagctgggactacggcatgtgccaccacacctggctaatttttgtatttttagtagaagacagggttttgccatgttgaccaggctggtcccgaactcctgacctcaagtgatccgcccaccttggcctctcaaaggaggccaagcctgtctggcctacaaagcagccttaaactttgactgtctggagtctccatggggttgtagtgttgtatctggtcccctccgtacccagagaacagtcagacctagagctctagacagatggcttgaaagttcctataactgtcatatttgcctttgcatgacaatacaaactcaacttttcagctaaaatttctttagagtgagccagtaaaacatacacagcccttgcttctagggtccagaaagcttctgttgaatgtggctcacgtgaggtagcccttagtgtcatggtttagaacatcctctggtgtcactcatacatgtgctggagaattgacctgtaatagctatataatcttggacagattttttaacttctgcaagcctcaatttcttcatctgtaaagggaggatgaggatgattagcttttaaggttgttagaagagttaaataatgcatttataatgcttactctggttcctggaactccataaatacaagatgacattatttattattatactccaaaaccacattcttttctactttaatatttctgccatcctcagccccctccctccctgcaccgcaccctgctttttctgcgttccaagtattctacattactttggtaactaaaaaacaaaagttcaataaatgtttgctagaaaaggaaagaaatttccaatttttttaaaataaaacttcaaagaacaatgtttatagggcaatgggaagttagttatagcctccatttttattaggggaaccaattgcaaaattgggttttctccacagttagaagagcagagtcattgccactgctcttttctcattgttccattagtggtagaaaggaagcccagtgatccccccatatgtctatctgaggcacagcagctggctcaggatgtaaagtaacttcttgtcatgtgcctttctaggcttctcttttgttgtttgaatttgaatggcttgagtatgaccaggggctctttcctatgtttcttctctaaaattctggtgttagcacgttagacctctcttttcccccttttttacttttagtgtgtctctcaccttggacattaacacagatactgtttagctctagtgctttggggtgaaagccaaagattttgttttttcctgtcctatgtgaatgttgctttatataataggtctgtttctcttaagtgtgcaaaataaatttttgtaaattgagctatacttaagatattgttaaagaaccatgaagtggctctttgtgtaatttgctgtgtaaaaatcttaatctgatttcttataagtttttctgcattttaactgcaacctgttcatcataatctaatagatagtggtttaagagcatagaatctgtagtcagactgcctgggttcaaatccaggctcttccatttagctaccagctttatgcttggttgtagatcctgttaagtaggaataatagtagttactttaaaaggttattgagaaaatctagagagttaatgcataaaaagtgtttagaatcatgcatggcaccttacaggtatttcttacatgtttggggctatcattacctagcatgggatttgtttaggacattgtgctagataatagagatccaaggagggtctgtagccaggttgctgcccacaagaggcttgtgggaacatagtttggttaaatcttacaaggacattggaagccttactctttttaagtggggatcatcccacacgccttaaatgctgccttgtccggttatcctcttgcaccaggagtgaggaaggagtgaattgggtggtggggtaaagatggccacctgggttcacacccccttcctctgctatatctttgcagtatgacttctctttggaaaagaaaaccattgagtgggctgaagagattaagaaaatcgaagaagccgagcgggaagcagagtgcaaaattgcggaagcagaagctaaagtgaattctaagagtggcccagagggcgatagcaaaatgagcttctccaagactcacagtacagccacaatgccacctcctattaaccccatcctcgccagcttgcagcacaacagcatcctcacaccaactcgggtcagcagtagtgccacgaaacagaaagttctcagcccacctcacataaaggcggatttcaatcttgctgactttgagtgtgaagaagacccatttgataatctggagttaaaaactattgatgagaaggaagagctgagaaatattctggtaggaaccactggacccattatggctcagttattggacaataacttgcccaggggaggctctgggtctgtgttacaggatgaggaggtcctggcatccttggaacgggcaaccctagatttcaagcctcttcataaacccaatggctttataaccttaccacagttgggcaactgtgaaaagatgtcactgtcttccaaagtgtccctcccccctatacctgcagtaagcaatatcaaatccctgtctttccccaaacttgactctgatgacagcaatcagaagacagccaagctggcgagcactttccatagcacatcctgcctccgcaatggcacgttccagaattccctaaagccttccacccaaagcagtgccagtgagctcaatgggcatcacactcttgggctttcagctttgaacttggacagtggcacagagatgccagccctgacatcctcccagatgccttccctctctgttttgtctgtgtgcacagaggaatcatcacctccaaatactggtcccacggtaagtcttttaaatcccccgccgactcccatattttcctgatgacagggattatgttttttcttgttgtttggttgatttttttcgagacagggtctcattctgttgcccaggctggaatgcagtggtgtgatcgtaccttattgtagcctcgacctcccgggctcaagcctgttgggacaacaggcacatgccatcacacctaatttttgtgttttttgtagagatgcggttttgacacgttgcccaggctggtctcaaactcctgacttcaagttatctgcctgccttggccttccaaagtgctgggattacaggtgtgagccaccaagactggcagtacagaacaacacattattgcattctagaatagaaatcaatagaagtcaagaaccttttgctacaccagcttacagatttcttttcctttcttttttttgagacagagtcttgctctgtcgcccaggctggagtgcagtggcaccatctcggctcactgccaacctccacctctcacgcaattctcctgcctcagcttcccgaatagctgggactacaggcatatgccaccacactcagctaatttttgtatttttagtagagacggggttttaccacgtttccgaggctggtctcaaactcctgaccttagttgatccacctgcctccacctcccaaattcctgggattacaggggtgagccgctgtgtccggccagcttacagattttttaatgtaaaagcacatcaagagactatttagagccaaaaaggagcctgagtatattggtgggatatctgtcagagaccataacaagcctgctattagctggtaaaaagatacaaaatcttggcctgctttacaagtcccagtaatagcattcttggtcaatgatagtctgtgtgaatgcttctattgtcagagagctcatagctgcttgtaaacttccataaaccttggcccgggtaaacttgatagagtgccgaacaccattattatcttcttggagtgggcttcattctaattcctcttggtcagttgttgggctctggtcagcactgtacttcttccttttcatggtactttgaatgctttagagaaattaaaatctccctggaaaagatcagggatccttggccaacactcccttcctctcatttaaagttagattaaccatttagagatcaaagccaataaactaacagcagagatctttctcaaactagaacgcttgtcattctttgaccagcctgatgatgggagtttctttctgacctgttcatctcaggatgggaagaggcctgcttcttgttggcttgagaatttgctgccttccacctgcaggtataaacagagctggagttctggtgtttcttttttcttttttttttttttgagacagattctaattctgtcacccaggctggaatgtagtggtgtgatctctgctcactgcaacctccacctcccagggtcgggcgattgtcctgcctctcgcagcctgccaagtagctgggattacaggcatgtgctatcgtgctcggctcatttttgtatttttagcagagatagggtttcgccatgttggccaggctggtctggaactcctgtgctcaagtgatccacccgcctcagcctcccaaagtgctgggattacaggtgtgagccacagtggctggccaattaaatattgactataatcaccctgttgtcttatcaaatggtagatcttattctatttttttctttgcttttttcttcaacagagtctcgctctgtcgcccaggctggagtgcagtggcgtgattttggctcactgcaacctctgcctcctgggttcaagggattcttctgcctcagcctccagagtagcagggattacaggcgcctgccaccatgcgcagctaatttttgtatttttagtagagacggggtttcaccatcttggccaggctggtcttgaactcctgacctcgtgatccgcccgcctcggcctcccaaatgctgggattacaggtgtgagtcaccgcacacagccattctttctattttttttgtacctattaaccaccagcccccgactactacccttctcagcccctgtggtaaccatccttctagtgtctatgtccatgggttcaattgctttgatttttagatcacacacataagtgagaacatgtgatgtttgtctttctatgcctggcttatttcacttaacataatgatctccagttccatccatgttgttgcaaatgacaggatctcattctttttcatggctgaacggtactccattgtgtacgagcaccacatttttttaatccatccttgtttaagttctttcactttactctttttttttttctttggagacagattttgctcttgttgttcaagctggcgtgcaatgttgcgatctcggctcactgcaacctccgcctcccgggttcaagtgattctcctgcctcagcctcccatgtagctgggattacaggcatgtgccaccacgcctggctaattgttctatttttagtacagacggggtttcatcatattggtcaggctggtctcaaactcttgacctcaggtgatgcacctgcctcggcctcccaaagtgctgggattataggtgtgagccactgcacctggcctatccattcatctcttgatggacacttaggttgcttccaaatcttggctattgtgaacagggctgcaacaaacatgagagtgcagatcttgtttatgttttactttctgggacatatccttaacttacaacttcaaaaaaaaactactaaatcttacaaccttttattttatttaatttatttttccccctgaacttgatagtaggtgattattttttaagagatgggatttttgtcccctaggctggggtgcagcttactgcctcagcctcctgtctgggactacaggtgtgcaccactatgcccagctgatttttgtagagttgggggtctcactgtttgcccaggctggtcttgaactcctgggctcaagtgatcctcctgcctcagcctcccaaagtgttgggattacaagcatgagccaccatgcccagcctaaatcttactaccttttaaatacaatttttactttatcatattttaaatttccaagtgctttgtctattccctcttttggtttggtgttttcatgctggtggctttggctttctattcacgtttaagtatgaggcactaaaaaaataacagtcacaaagtttgtgtgtcagaggtgtgcttgctgatcattaagcttcactataggttgaaagttaattggctttgccggggaagtccccaaaatgtaaacatgtgaagggcttgggtgagggtttctccagagaattctggctgaggactgatgttggggaatgctggtgtgttgcttttacttaatccttgtttaagttctttcacttcactctgccccacactgtgcctggtgcttagtcctctttggtctctggttcccattttctggagactataccttctgcctcctgcacagggtggggatgaatggggactgtttcaccaagggctttactgctctgatcactgactgtgtgtatctttgccttatcccaacagtacctggtgcttccagagtctgagcccctcttagggatcctaatgggctgatagactgactcccccctttcctacttacttacttagtttataacttcattgctggcaattaccatcccttcattatctttccacattctaaaatttcattgaaatatatcacaccagtaatcccagcattttgggaggccgaggtgggaggattgcttgagcccaggacttcgagatcagcctgggcagcatactgagatcttgtctttgtttctttatttaaaaagaataagaaatatattgtccactgctaccccctttccttttattgctttaaagatgaatacagttttaattctttatttttaaatttttattattttttttgagacagggtcttgctctgtaatcacggctcactgcagcctcgacctcctgggctcaagtgatcctcctgcctcagccttctgggtagctgggactacaggcacgtgccaccacacctggctaactttttgtgttttgtagagatgagtctcaactatgttgtccaggctggtctcaaaattctgagcttaagcagtccttctgcctcagcctcccaaagtgctgggattataggcatgagccattgctcctggccttagtttgtttatgatcattttagtgggcttttgggaagaagagatggtaaagacattttagtgtgttaaatgaaattctccctttgaaaattctaatggacttcaatttattccacaactgttgatgaagaccagtaagtgtaaagccaagagtcaggccctggggatttagaaatgggcatgacatggtaactgacctctctgcaagctcacactaatgtaatgtgataagtactttgccagcagtgtgactaaagctctgtgggaccacaggtggcagtgtgtccgctgagttgtgcacacacattagcagtgggttaaaaggaggtttatggaaagacttggtagaagctgtcttgaggttaggcttgagggccacataggtacagaaaggactgccaccttctgaggcctgagagcatgctgagactcttcagaagcttgagcactaatatttgtctcctttcacaaacgtactttccctcaaacactcatatttaatttggaaagatttcataataattctcatttttgtctagtttctacttggttacaaatatggagcacttcagtcatcgagtatgaaaatccctttttatgccagagtcaacctttgcccctgcaggaagtaaattttactattcttttattgaaaaaataatgacaaatggctaccatgaatgcagttaatggtgtttaaaaagtcatatctagattactcctaagagccttatatatatttgaaaatggtaccctggagtaaataaatttggtgagtaactggagaaactaaacactgttataattttgatgagggatcctttcaaaccttgtctgtccgtttggggtctgctggattagcctgttgcctgtttgattcatgaaacatttatatgaaggtgacatcttattttgtttgtccctgttcaagaaaagtatcatctgttaggatagatgatacttttaaatcatccatttaaaatcagtcatttaatatcccgtgcatctttaaaaaatattgtagagcatgtacaattaataaatatatctttttttcttatttatttatttatttagagacggtgtctcgctgtgtcaaccaggctggagtgcagtggcgcaatcttggcccactgagacctccgccttctgggttcaagtgatcctcctacctcagcctcccaagtagttgggattacaggaatgcaccaccacacttggcccattttgtatttttagtagagatggggtttctccgtgttggtcaggctggtcttgaactcccgacctcaggtggtctgcctgccttgccccccaaagtgctgggattacaggtgtgagacacctcacccgctgttttttcacttaattcagatgctttaaataattaacagtcgtatgttcattatttgctaacctaacaccctgtgctggatatttggtaggttgtccctaaaatttattacaaatgatgttgagatggatatattcctatacataaatattggtttgcattattattatttcttcagactagtttcctagaaataggtaactggatcaaagaatctgagcagttttctggatttatgccatgtttacctaaagcataccaaataacatttccaccagtagggtatgtgagagtactgcatgtatgtttaaaatagatacgatttttttttttttcagacagtctcactctgtcacccaggctggagtgcaatggcatggtctccactcactgcaacctccacctcccgggttcaagcgattttcccacctcagcctcccgagtagctgggactacaggcatgtgccaccacacccggctgatttttgtatttttagtagagatggggtttcactatgttggccaggctggtctcaaactcctgacctcatgatccacctgcctcggcctcccaaagtgctgggattacaggcgtgagccaccgcacctggctagatatgattttattaacatttgtaatgtgcagtattctgggtgtatgcctgcatcccaccaggcatttaggaggcattatgagaagggcaggcagttgaagaatagtcctgactccttcctggctggggaatgtcatgtgagagctggcattgctctgtggctgttggtttctttagcccaggttaaagcccgtctctaaagagggagacacaatcttagacctgccaagtggctagactggtgtgatccaatccctgtgccttattgtgactcttgtaccataatgacagccctgtgcttgggctgctgagtgtgtgctgtgggtgagatggttaagcaccaacagaatgggctctgaggcctatagccatcctggctttccccagcagactttgaggagagcatctggttacatctgtataatttcgaacttgcttctaggacagagggcttcctaccacctaaccatgctccctctccccaccaataaaacaaggaaataaaaaaatttttgtgtgaacatcttccctggttcttgggacctgcgccaagctgtaaaagggcttgtgacctcgttcctgtggtcagtgggtcctttggcctgggagtgtgctgtcttcctgccttgggcttgatttgtgatcagaagtggtgggggagttaatcacggcactctgtacgcctatccgactggggttagtaagttccagtggcagggagcaggctgcctgtagctggtgggcaggtccctagagtccagattccagtctgtgggatccagcctggtgtggctagtatgccttctgtcctggcactgtaggatggcagctttggcaggaaacgaagcacagtacatgatgtagggcaaaggtgtggagtaccctcaaaaccctgggttcctgagctgtccaggttcctaagctgtccatgcatgggaaccacactcacagcctctgccgaagagtagagactttgggatagagttagctccaaagtgggagtatttgaagtgaaaaatgatgcctcagaactgatacttcagcctgggaaagggataatgctggagagaaatcgcagccagcttggacatcagtcccgggggcagccacagcttctttcttcaactcaaatcctggagatttgggcatgcaagggcctgtcgtgttgtggcgctttctcaatcaccaaagtgggactagggcagagcaggacagccacaattgcaggtgacctcccacctttttccctctcagctggcactcccctacccctctttgttccttttcagggtgtctgaccagcaacctggcgatttcttccaacctgaccgcttttcttggggcttgaatagtgtcagaaatgccaacccctggactaggctgcctcagtgacttctttgggggcccaggtcttcttgccttaatcttcttgtttttccactaggtcacccctcctaatttctcagtgtcacaagtgcccaacatgcccagctgtccccaggcctattctgaactgcagatgctgtcccccagcgagcggcagtgtgtggagacggtggtcaacatgggctactcgtacgagtgtgtcctcagagccatgaagaagaaaggagagaatattgagcaggtgagcggttggtcagccaggagggcaggctcagacctgtggagctggagtagtgtcctcccctgtcctagagcccctcagacttgtagcaccaacctcctttcctgtgggtttgtgaggacacggggcatgttttggggaaagccagcagccttgggagcagtacttcttttggaagctattccgggacccaggctgagagacagctttgggagaattggagctgttctccaactaccccctcctttctctgcctctccaaaaggggcagagctaggcttggggtatggtcaaatgtctctttccaggaattttcatcctgtcatctatgcccagagtgttgaattagggtactggagtgttagaccttggtgacattttctgacttgggctgttttcttagagtagccgcctgcccaggctgtggagttgaccattctggtgggcgtgtcagctgtggctgcaactcctttggaaaatgcctgctcctgtttagaaggctgtgcctgtgttcattaaagagtcactggggtcctgttgtgttggacacagtcaaggcacaaaatcaaatcctatccagtatctgacggcctgggtggggcggagaacggacttcacacactagtttgttgaggtctcttggaaagcacagcaaagagcagcagtaggtgctaaaccccttgtttcttttcttagattctcgactatctctttgcacatggacagctttgtgagaagggcttcgaccctcttttagtggaagaggctctggaaatgcaccagtgttcagaagaaaaggtgggaatcttcatgtttcttgggaaccctctggaaaaggagggaccaagtatccatcctggttttctcccttggcccacacacaacctttttgctttgccagtgactacaggtacaagtatttgaagggcagaatctcccaagtattcagacaggccgaggcctgcgtttggcttggtggttgaagcattgggttgaaaacttcagaatcccagtgaagggaagggaatattggcctgggctaaaagccaaacttcttagatgattagggagggagattgctggactttttgcctagactgctctttgaagagcagccatttctgggttcagtatccttccttggtgggaagatggatgtgggtggtgggacccagacagagctcagcaacagtgagctgggactgggctgactcctgaaggttgatggccgtcaccaaacccgtttgccttggcttccaccacagcccacctctgcttgctaggggaccttatggagactcacggggctctgggggaaggatgtagacattctgctctcccccagtccccgtcccacacacacactccttcactcagctgtgctgtgaccccacccttttctttaatctgtgttctcttctctcccttagatgatggagtttcttcagttaatgagcaaatttaaggagatgggctttgagctgaaagacattaaggaagttttgctattacacaacaatgaccaggacaatgctttggaagacctcatggctcgggcaggagccagctgagaccaggccctgcctaggccctgccgcagaaccaccatccctgggaggccctgcagagcccacctgtggggaaagagaaggggcagcttccggattttcttttgggggttagaaggtcaggtgtggagactgctcgccagtctctgtgagcctaggccctgagctggggaggtggggaagattcgggcatgtgagtgcccccagaactgtcctggctccttccgtattaaacgcatttgcattttgagaagtgtccttcccacttcagccctccggagagactaccctagtctttctggggtgtttatgtcctcagctgaagcctggcctagttgctgagaggggctggggagatggggcgggagggccagactcagtgctgctgtggagctaggtgcttcccccttcccctgagactggtggactgaactccagtcaagttgagttcaagtgaaagattcttccagggttttattttttcccctcctaacaaagtctcatagtgttaacactggttctgcaatatctctgaggtgcaaagaatgcacttttccctatggggcccagagtttgccttttctgccaggcagtcaccatgcttccctaccccagcctgtttcttttggcttggtttggaccacagtcctctgctacccagggttttagagcccctgctctaggaaacagtttaagaaatcattggccccttcccagcacattgaatgggtaagcagacaggccatgatttagttggccagcactaactccacctctgttctccttgaacagcttcccctccagcccactgctttaggatgacacaatgaataacacctagtcatagaaatcagtctctctggtttgttttgtattatgttgtacatcattaaagatctaaatacaaaggatatacagtcttgaatctaaaataatttgctaactaactattttgattcttcagagagaactactaataaaaatctaaaaggtatgtcttggtgtgataacttctttattaccaaggctgttttatgggagtgggggggccatgtggtgtataaagagatttcttgcttagatttgctggggatagaataggacctaaccgttttctatttcggagctttcttcccacttcagacctgtcctcgtttttggtttgcaaaggcaagcctagactgagtcttttaaacctggcccagctccgtctgtggaccaaggacgtaggccttcctgactgtggagttgggataggctgggccttcagggggatcacccagtaaaagaggaagaagcccttttactgtgagcagcaggcatttatgcatcatttgccacaagcagaggatgggatttaaactgtttcaccagctctcagtggtgctgttgtatatgatgatagaatagggttcattcctaaagtaagtaagtttttgtccttgaccttaatactgtgccattgtcccacttgagcttttggctagactgggtctttgaaggtttttagtgatcatgtgagggaggggccgcagtgccctgcagccgggaagcaccaataaggatgccccctggcggtcagatgaggcactgtcaggctgacaaagggtgactggaccaggctgctagtgtctcaagagagccatttaaccctcttcttggccttttaattaagccctccttttaaaaccgtaaaaagttatcctttatctgaaggaaataaaggctcacatagtaatgggcgttcgggccaaggccgtgactcatgaatctccatttgacccagagtggctttaaagtaattggcacctggaggctactttgtcttccctgtcctgagctgcttcctgctaaaaagatacacagcaggcagaacttggctgaaggtttgtgtgtgaattctgtttcagaggcctcagactgttgaacagagcactctgttcaactgccaaggttcccaacacagacccctgagaggcagggtggctgggcag

SEQ ID NO: 4 - DNA, 19 bp, Artificial Sequence
  Synthetic Primer
ccacaatgccacctcctat

SEQ ID NO: 5 - DNA, 18 bp, Artificial Sequence
  Synthetic Primer
agaataggcctggggaca

SEQ ID NO: 6 - protein, 8 aa, Artificial Sequence
  Synthetic Primer
KVLSPPHI

SEQ ID NO: 7 - DNA, 24 bp, Artificial Sequence
  Synthetic Primer
aaagttctcagcccacctcacata

SEQ ID NO: 8 - protein, 8 aa, Artificial Sequence
  Synthetic Primer
KVLMRPTS

SEQ ID NO: 9 - DNA, 24 bp, Artificial Sequence
  Synthetic Primer
aaagttctcatgaggcccacctca

SEQ ID NO: 10 - protein, 8 aa, Artificial Sequence
  Synthetic Primer
ATKQKVLS

SEQ ID NO: 11 - DNA, 24 bp, Artificial Sequence
  Synthetic Primer
gccacgaaacagaaagttctcagc

SEQ ID NO: 12 - protein, 4 aa, Artificial Sequence
  Synthetic Primer
KVLS

SEQ ID NO: 13 - DNA, 24 bp, Artificial Sequence
  Synthetic Primer
gtgccacgaaacaaagttctcagc

SEQ ID NO: 14 - protein, 9 aa, Artificial Sequence
  Synthetic Primer
NPILASLQH

SEQ ID NO: 15 - DNA, 27 bp, Artificial Sequence
  Synthetic Primer
aaccccatcctcgccagcttgcagcac

SEQ ID NO: 16 - protein, 9 aa, Artificial Sequence
  Synthetic Primer
NPIPRQLAA

SEQ ID NO: 17 - DNA, 27 bp, Artificial Sequence
  Synthetic Primer
aaccccatccctcgccagcttgcagca

SEQ ID NO: 18 - protein, 9 aa, Artificial Sequence
  Synthetic Primer
LASLQHNSI

SEQ ID NO: 19 - DNA, 26 bp, Artificial Sequence
  Synthetic Primer
ctcccagcttgcagcacaacagcatc

SEQ ID NO: 20 - protein, 4 aa, Artificial Sequence
  Synthetic Primer
LASL

SEQ ID NO: 21 - DNA, 15 bp, Artificial Sequence
  Synthetic Primer
ctcgccagcttgtag

SEQ ID NO: 22 - protein, 10 aa, Artificial Sequence
  Synthetic Primer
SKSGPEGDSK

SEQ ID NO: 23 - DNA, 30 bp, Artificial Sequence
  Synthetic Primer
tctaagagtggcccagagggcgatagcaaa

SEQ ID NO: 24 - protein, 10 aa, Artificial Sequence
  Synthetic Primer
SKSGPDQRAI

SEQ ID NO: 25 - DNA, 30 bp, Artificial Sequence
  Synthetic Primer
tctaagagtggcccagaccagagggcgata

SEQ ID NO: 26 - protein, 9 aa, Artificial Sequence
  Synthetic Primer
SGPEGDQKM

SEQ ID NO: 27 - DNA, 27 bp, Artificial Sequence
  Synthetic Primer
agtggcccagagggcgatagcaaaatg

SEQ ID NO: 28 - protein, 4 aa, Artificial Sequence
  Synthetic Primer
DSKM

SEQ ID NO: 29 - DNA, 27 bp, Artificial Sequence
  Synthetic Primer
gtggcccagagggcggatagcaaaatg

(19) United States
(12) Patent Application Publication
(10) Pub. No.: US 2022/0177527 A1
(43) Pub. Date: Jun. 9, 2022

(54) METHODS OF DETECTING AND TREATING HEREDITARY SPASTIC PARAPLEGIA

(71) Applicant: UNIVERSITY OF MIAMI, Miami, FL (US)

(72) Inventors: Stephan L. Zuchner, Pinecrest, FL (US); Mohammad A. Faghihi, Miami, FL (US)

(21) Appl. No.: 17/439,338

(22) PCT Filed: Mar. 13, 2020
(86) PCT No.: PCT/US20/22716

§ 371 Date: Sep. 14, 2021

Related U.S. Application Data
(60) Provisional application No. 62/819,217, filed on Mar. 15, 2019.

(51) Int. Cl. C07K 14/435; A61P 25/00

(52) U.S. Cl. CPC C07K 14/435; A61K 38/00; A61P 25/00

(57) ABSTRACT

The present disclosure relates to methods of detecting and, optionally, treating Hereditary Spastic Paraplegia and other neurological diseases.

### CROSS REFERENCE TO RELATED APPLICATION

This application claims the benefit of U.S. Provisional Patent Application No. 62/819,217, filed on Mar. 15, 2019, the entire contents of which is fully incorporated herein by reference.

### STATEMENT OF FEDERALLY SPONSORED RESEARCH

This invention was made with government support under grant number NS072248 awarded by the National Institutes of Health (NIH). The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 54103_Seqlisting.txt; Size: 109,7669 bytes; Created: Mar. 13, 2020.

### FIELD OF THE INVENTION

The present disclosure relates to methods of detecting and treating Hereditary Spastic Paraplegia (HSP) and other neurological diseases.

### BACKGROUND

Hereditary spastic paraplegia (HSP) represents a group of genetically highly heterogeneous rare inherited neurodegenerative diseases, characterized by the pathological hallmark of a length-dependent degeneration of corticospinal tract axons. ^{1,2} Clinically, HSPs are marked by progressive spastic paraparesis, although the clinical presentation encompasses a wide spectrum of phenotypes. In pure forms of HSP, progressive spasticity and weakness in the lower extremities are common features. In complex forms of HSP additional clinical symptoms include cataracts, ataxia, epilepsy, cognitive impairment, peripheral neuropathy, optic neuropathy, and deafness. ^{1,2} The prevalence of HSP has been estimated at 1.3-9.6 in 100,000.¹⁻³ Thus far, at least 58 genes have been reported to cause HSP in a Mendelian fashion.⁴ Yet, approximately 40% of affected persons are still not diagnosed even after whole-exome sequencing (WES). Further, many of the genes reported in recent years have only been described in few families.⁵

### SUMMARY

The disclosure provides a method of detecting neurological disease, such as Hereditary Spastic Paraplegia. The method comprises detecting the presence of a truncating mutation in a Ubiquitin Associated Protein 1 (UBAP1) gene in a biological sample from a subject. Optionally, the method comprises treating the neurological disease (e.g., Hereditary Spastic Paraplegia) in the subject. In this regard, the method further comprises administering to the subject a composition that comprises an agent selected from the group consisting of a UBAP1 peptide; polynucleotide that encodes a UBAP1 peptide; an agent that induces expression of an endogenous nucleotide sequence that encodes a UBAP1 peptide; an agent that blocks expression of a mutant UBAP1 gene; and an agent that corrects the mutation in UBAP1 gene. In various aspects, the agent comprises a UBAP1 peptide, an expression vector comprising a polynucleotide encoding the UBAP1 peptide, a UBAP1 antisense oligonucleotide, a CRISPR Cas9 protein and one or more guide RNA molecules, or combinations thereof.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment,” “some embodiments,” “various embodiments,” “related embodiments,” each of these types of embodiments is a non-limiting example of a feature that is intended to be combined with any other feature, or combination of features, described herein without having to list every possible combination. Such features or combinations of features apply to any of the aspects of the invention.

The headings herein are for the convenience of the reader and not intended to be limiting. Additional aspects, embodiments, and variations of the invention will be apparent from the Detailed Description and/or drawings and/or claims.

### BRIEF DESCRIPTION OF THE DRAWINGS

FIG. 1. Pedigrees of HSP families with UBAP1 truncated mutations. All pedigrees suggest an autosomal dominant or a de novo Mendelian trait. HSP affected persons are marked by filled symbols, individuals with unclear affection status are marked by a question mark. ‘mut’ depicts the presence of a causative allele. Sanger traces exemplify the confirmation of variants detected via next-generation sequencing. The penetrance of truncating UBAP1 variants is reduced: Individual F5-III.2 was subjectively unaffected at age 14 but showed brisk reflexes of lower limbs, indicating potential dysfunction of the corticospinal tract. The 80-year-old grandfather of the index case in family 9 (F9-III.1) was unfortunately not available for a neurological examination but reported to be in good health and without any indication of a gait disturbance.

FIGS. 2A-2B. The structure of UBAP1 and affected person mutations. FIG. 2A) Schematic diagram showing all exons and UTRs of UBAP1 based on gene model NM_016525.4. The grey boxes represent the coding sequence of UBAP1. All variants occurred in exon 4 of UBAP1. UMA protein domain includes amino acids of 17-63 and two UBAs include amino acids 389-430 and 451-489. All truncations are listed below and clearly depicting the preserving of the UMA domain, but loss of the two UBA/SOUBA domains. FIG. 2B) Western blot analysis with an antibody recognizing amino acids 25-75 of UBAP1 showing a notable decrease in the amount of full-length UBAP1 in fibroblasts of the affected affected persons from three different families compared to control fibroblasts. Truncated UBAP1 protein was detected in examined affected person fibroblasts at the predicted sizes, but not in control fibroblasts.

FIGS. 3A-3D. Functional in vitro and in vivo studies of truncated UBAP1 protein. FIG. 3A) Immunostaining of U2OS cells transfected with HA-WT-UBAP1, HA-Fs-UBAP1 (p.Leu121Profs*18) and VPS-28-Myc. Both wild-type and mutant UBAP1 co-localize with VPS28-Myc. FIG. 3B) Co-immunoprecipitation assay shows protein-protein interaction between both HA-WT-UBAP1 and HA-Fs-UBAP1 with VPS28-Myc. Ubiquitinated proteins co-immunoprecipitated with HA-WT-UBAP1, but not with HA-Fs-UBAP1. FIG. 3C) Motor neuron axons in Tg(olig2::DsRed) zebrafish embryos at 48 hpf. Embryos were injected with CRISPR Cas9 and sgRNAs against UBAP1 supplemented with human RNA rescue of wild-type or truncated mutant UBAP1. Truncated and misshaped axons were more commonly observed with mutant hRNA rescue (indicated by asterisks). Scale bars, 50 μm. Phenotypic difference between treated groups was evaluated by Fisher exact test. Samples were assigned to either normal or affected categories based on the presence of truncated and misshaped axons. The Fisher exact statistic value was determined to be 0.003; the result is significant at p<0.005. F0 CRISPR+Wild-type hRNA rescue, normal, n=11; affected, n=1; F0 CRISPR+mutant hRNA rescue (family 4, p.Leu121Profs*18), normal, n=9, affected, n=15. FIG. 3D) Quantification of the individual motor axon lengths. P values were calculated with one-tailed Student's t test, p=0.0008, n=9 (each).

FIG. 4. RT-PCR of a patient (Family 1) and two controls (C1, C2) from fibroblasts indicate that truncated UBAP1 mRNA escapes nonsense mediated decay.

FIGS. 5A-5B. FIG. 5A) Homo sapiens Ubiquitin Associated Protein 1 (UBAP1) polynucleotide sequence, derived from NCBI GenBank: CR533551.1 (SEQ ID NO: 1). FIG. 5B) Homo sapiens Ubiquitin Associated Protein 1 (UBAP1) amino acid sequence, derived from NCBI GenBank: CAG38582.1 (SEQ ID NO: 2).

### DETAILED DESCRIPTION

The disclosure provides methods of detecting (and, optionally) treating Hereditary Spastic Paraplegia and other neurological diseases. In various embodiments, the neurological disease is peripheral neuropathy, spinocerebellar ataxias, motor neuron disease or amyotrophic lateral sclerosis (ALS). The method comprises detecting the presence of a truncating mutation in a Ubiquitin Associated Protein 1 (UBAP1) gene in a biological sample from a subject. The genomic DNA sequence of UBAP1 is provided as SEQ ID NO:3. A “truncating mutation” or “truncating variant” refers to a DNA or RNA nucleotide change that causes a premature open reading frame stop or shift leading to an early termination of protein translation of the reference UBAP1 mRNA. Examples include nonsense changes that cause a “stop codon”, insertions, deletions, copy number changes, chromosomal rearrangements, and splice altering nucleotide changes. In various aspects, the truncating mutation in UBAP1 is c.436_437insTGAG, c.426_427delGA, c.382del, c.361dupC, c.373C>T, c.373C>T, c.286_290dupCCAGA, c.295dupG, c.1091delC, c.426_427delGA, or combinations thereof. Optionally, the method comprises diagnosing the subject with neurological disease (such as HSP) when the presence of a mutation in the UBAP1 gene is detected.

Optionally, the method further comprises administering to the subject a composition that comprises an agent selected from the group consisting of a UBAP1 peptide; polynucleotide that encodes a UBAP1 peptide; an agent that induces expression of an endogenous nucleotide sequence that encodes a UBAP1 peptide (for example, an antisense oligonucleotide); an agent that blocks expression of a mutant UBAP1 gene (for example, by acting on the DNA or RNA mutation or on a nearby cis variation); and an agent that corrects the mutation in UBAP1 gene (for example, a CRISPR guided nucleotide template or a prime editing guided nucleotide template). In various aspects, the agent comprises a UBAP1 peptide, an expression vector comprising a polynucleotide encoding the UBAP1 peptide, a UBAP1 antisense oligonucleotide, a CRISPR Cas9 protein and one or more guide RNA molecules, or combinations thereof.

The diagnostic gap for rare neurodegenerative diseases is still considerable, despite continuous advances made in gene identification. Many novel Mendelian genes are only supported by few families worldwide. Disclosed herein is the identification of an autosomal-dominant gene for hereditary spastic paraplegia (HSP) in eight families of diverse geographic origin, all carrying unique truncating changes in a circumscript region of UBAP1 (Ubiquitin Associated Protein 1).

### Hereditary Spastic Paraplegia (HSP)

HSP is a neurodegenerative disease characterized by progressive lower limb spasticity and weakness, as well as frequent bladder dysfunction. At least 40% of patients are currently undiagnosed after exome sequencing. HSP represents a group of genetically highly heterogeneous, rare, inherited neurodegenerative diseases, which are characterized by the pathological hallmark of a length-dependent degeneration of corticospinal-tract axons.² Clinically, HSPs are marked by progressive spastic paraparesis, although the clinical presentation encompasses a wide spectrum of phenotypes.

The disclosure provides a method of detecting Hereditary Spastic Paraplegia (HSP) in a mammalian subject, the method comprising detecting the presence of a truncating mutation in a Ubiquitin Associated Protein 1 (UBAP1) gene in a biological sample from the subject. Optionally, the method comprises administering to the subject a composition that comprises an agent selected from the group consisting of a UBAP1 peptide; polynucleotide that encodes a UBAP1 peptide; an agent that induces expression of an endogenous nucleotide sequence that encodes a UBAP1 peptide; an agent that blocks expression of a mutant UBAP1 gene; and an agent that corrects the mutation in UBAP1 gene.

### Ubiquitin-Associated Protein 1

The endosomal sorting complex required for transport (ESCRT) machinery facilitates the lysosomal degradation of ubiquitinated cell surface receptors. Ubiquitin associated protein 1 (UBAP1) is a subunit of human ESCRT-I¹². UBAP1 is also known in the art as the following aliases: UAP, UBAP, NAG20 or SPG80. The protein is essential to sort endocytic ubiquitinated cargos into multivesicular bodies (MVBs).¹¹ It also plays an important role in proteasomal degradation of ubiquitinated cell-surface proteins, including EGFR (Epidermal growth factor receptor) and BST2 (Bone Marrow Stromal Cell Antigen 2).¹¹

Disclosed herein is the identification of pathological truncating variants in UBAP1 in HSP patients from Iran, USA, Germany, Canada, Spain, and Bulgarian Roma. The genetic support ranges from linkage in the largest family (LOD=8.3) to three confirmed de novo mutations. It is shown herein that mRNA in patient fibroblasts escapes nonsense-mediated decay leading to expression of truncated protein; in addition, full length protein levels are reduced compared to controls. This suggests either dominant negative effect or haploinsufficiency. UBAP1 links endosomal trafficking to the ubiquitination machinery, and linking both pathways directly provides a bridge towards a more unified pathophysiology.

UBAP1 protein has two main domains. The UMA (UBAP1-MVB12-associated) domain in the N-terminal region (17-63 aa), which mediates the association with the ESCRT-I complex, and a solenoid of overlapping ubiquitin-associate UBAs (SOUBA) domain in the C-terminal region (389-498 aa).^{11,12} Both domains allow UBAP1 to act as a molecular bridge connecting the endosomal trafficking pathways to the ubiquitination machinery. To decipher the pathophysiology of UBAP1 in HSP it was noted that all but one of the identified changes fall within a circumscript area of the protein, between Asp99 and Ser 146, with the change in family 9 at Pro364 being the only outlier (NM_016525.4). The amino acid sequence of UBAP1 is shown in SEQ ID NO: 2.

### Detecting Mutations in UBAP1 Gene

In various embodiments, the method of the disclosure comprises detecting the presence of a truncating mutation in a Ubiquitin Associated Protein 1 (UBAP1) gene in a biological sample from a subject.

In various embodiments, the truncating mutation detected in UBAP1 is c.436_437insTGAG, c.426_427delGA, c.382del, c.361dupC, c.373C>T, c.373C>T, c.286_290dupCCAGA, c.295dupG, c.1091delC, c.426_427delGA, or combinations thereof. Mutations in the UBAP1 gene may be detected using next-generation sequencing, such as a gene panel, whole exome sequencing (WES), whole-genome sequencing (WGS), Sanger sequencing, and related DNA sequencing methods, polymerase chain reaction (PCR), real-time PCR (RT-PCR), microarray or Multiplex Ligation-dependent Probe Amplification (MLPA) assays for sequence and copy number variants, DNA restriction enzyme digestion and gel electrophoresis, and other DNA mutation detection methods known in the art. Truncated, mutant UBAP1 may also be detected via next-generation sequencing or PCR amplification of UBAP1 mRNA or western blotting of UBAP1 protein.

The sample may be any biological sample taken from the mammalian subject, including, but not limited to, any tissue, cell, or fluid (e.g., blood) which can be analyzed for a parameter of interest, such as the presence or amount of a nucleic acid (e.g., UBAP1 mRNA) or a protein (e.g., UBAP1 protein). In various embodiments, the biological sample is a plasma, saliva, urine, or skin sample.

### Agents

The method optionally comprises administering to the subject a composition that comprises an agent selected from the group consisting of a UBAP1 peptide; a polynucleotide that encodes a UBAP1 peptide; an agent that induces expression of an endogenous nucleotide sequence that encodes a UBAP1 peptide; an agent that blocks expression of a mutant UBAP1 gene; and an agent that corrects the mutation in UBAP1 gene. The UBAP1 protein is described above. In various aspects, the method comprises administering to the subject the UBAP1 protein (SEQ ID NO: 2), or a protein that is at least about 60%, at least about 65%, at least about 70%, at least about 75%, at least about 80%, at least about 85%, at least about 90%, at least about 95%, or at least about 96%, or at least about 97%, or at least about 98%, or at least about 99% identical to SEQ ID NO: 2.

In various embodiments, the method involves administering to a mammalian subject a composition comprising a polynucleotide that encodes a UBAP1 peptide. Polynucleotides are typically delivered to a host cell via an expression vector, described in more detail below. In various embodiments, gene expression constructs comprise the polynucleotide sequence of UBAP1 (SEQ ID NO: 1) or a polynucleotide sequence that is at least about 60%, at least about 65%, at least about 70%, at least about 75%, at least about 80%, at least about 85%, at least about 90%, at least about 95%, or at least about 96%, or at least about 97%, or at least about 98%, or at least about 99% identical to SEQ ID NO: 1 (or any known wild type UBAP1 nucleic acid sequence). In various aspects, the polynucleotide sequence administered to the subject encodes the UBAP1 protein (SEQ ID NO: 2), or encodes a protein that is at least about 60%, at least about 65%, at least about 70%, at least about 75%, at least about 80%, at least about 85%, at least about 90%, at least about 95%, or at least about 96%, or at least about 97%, or at least about 98%, or at least about 99% identical to SEQ ID NO: 2.

In various aspects, the agent administered to the subject is an agent that blocks expression of a mutant UBAP1 gene. Representative agents that block expression of a mutant UBAP1 gene (or, put another way, reduce the production of mutant UBAP1 protein) include antisense oligonucleotides (small RNAs) targeting UBAP1. The terms “small RNA” or “small RNAs” as used herein, refer to small RNAs known to trigger RNAi processes in mammalian cells, including short (or small) interfering RNA (siRNA), and short (or small) hairpin RNA (shRNA) and microRNA (miRNA). Small RNAs are <200 nucleotides in length, and are typically non-coding RNA molecules. Small RNAs are typically designed to target one or more regions of the UBAP1 gene or non-coding elements associated with the UBAP1 gene (e.g. promoter or miRNAs associated with UBAP1 gene expression). In various embodiments, the binding of the small RNA to the UBAP1 gene will induce its degradation and therefore interferes with UBAP1 gene silencing and/or UBAP1 protein expression levels.

As an understanding of natural RNAi pathways has developed, researchers have designed artificial shRNAs for use in regulating expression of target genes for treating disease. Several classes of small RNAs are known to trigger RNAi processes in mammalian cells, including short (or small) interfering RNA (siRNA), and short (or small) hairpin RNA (shRNA) and microRNA (miRNA), which constitute a similar class of vector-expressed triggers [Davidson et al., Nat. Rev. Genet. 12:329-40, 2011; Harper, Arch. Neurol. 66:933-8, 2009]. shRNA and miRNA are expressed in vivo from plasmid- or virus-based vectors and may thus achieve long term gene silencing with a single administration, for as long as the vector is present within target cell nuclei and the driving promoter is active (Davidson et al., Methods Enzymol. 392:145-73, 2005).

In various aspects, an agent is administered that corrects the mutation in UBAP1 gene (or enhances expression of the wild-type UBAP1 gene or reduces expression of a mutant UBAP1 gene). Modification of the host genome to accomplish these goals may be a performed using genome editing. Genome-editing techniques using, e.g., designer zinc fingers, transcription activator-like effectors nucleases (TALENs), or CRISPR-Cas (clustered regularly interspaced short palindromic repeats-CRISPR associated) systems are contemplated for producing targeted genome modification.

Zinc-fingers nucleases (ZFNs) and Transcription activator-like effector nucleases (TALENs) are customizable DNA-binding proteins that comprise DNA-modifying enzymes. Both can be designed and targeted to specific sequences in a variety of organisms (Esvelt and Wang, Mol Syst Biol. (2013) 9: 641, which is incorporated by reference in its entirety). ZFNs and TALENs can be used to introduce a broad range of genetic modifications by inducing DNA double-strand breaks that stimulate error-prone nonhomologous end joining or homology-directed repair at specific genomic locations. The versatility of ZFNs and TALENs arises from the ability to customize the DNA-binding domain to recognize virtually any sequence. These DNA-binding modules can be combined with numerous effector domains to affect genomic structure and function, including nucleases, transcriptional activators and repressors, recombinases, transposases, DNA and histone methyltransferases, and histone acetyltransferases. Thus, the ability to execute genetic alterations depends largely on the DNA-binding specificity and affinity of designed zincfinger and TALE proteins (Gaj et al., Trends in Biotechnology, (2013) 31(7):397-405). The following U.S. granted patents, incorporated by reference, describe the use of ZFNs and TALENs in mammalian cells, U.S. Pat. Nos. 8,685,737 and 8,697,853.

CRISPR-Cas (clustered regularly interspaced short palindromic repeats-CRISPR associated) is an RNA-mediated adaptive immune system found in bacteria and archaea, which provides adaptive immunity against foreign nucleic acids (Wiedenheft et al., Nature (2012) 482:331-8; Jinek et al., Science (2012) 337:816-21). However, multiple research groups have demonstrated how the biological components of this system can be harnessed to introduce directed modification to the genome of mammalian cells (Cong et al., Science (2013)339:819-23; Mali et al., Science (2013) 339(6121):823-6; Jinek et al., Elife. (2013); 2:e00471). CRISPR-Cas systems are generally defined by a genomic locus called the CRISPR array, a series of 20-50 base-pair (bp) direct repeats separated by unique “spacers” of similar length and preceded by an AT-rich “leader” sequence (Wright et al., Cell (2016) 164:29-44).

Three types of CRISPR/Cas systems exist, type I, II and III. The Type II CRISPR-Cas systems require a single protein, Cas9, to catalyze DNA cleavage (Sapranauskas et al., Nucleic Acids Res. (2011) 39(21): 9275-9282). Cas9 serves as an RNA-guided DNA endonuclease. Cas9 generates blunt double-strand breaks (DSBs) at sites defined by a 20-nucleotide guide sequence contained within an associated CRISPR RNA (crRNA) transcript. Cas9 requires both the guide crRNA and a trans-activating crRNA (tracrRNA) that is partially complementary to the crRNA for site-specific DNA recognition and cleavage (Deltcheva et al., Nature (2011)4 71(7340):602-7; Jinek et al., Science (2012) 337:816-21).

Other publications describing the CRISPR systems and Cas9, include the following Cong et al. Science (2013) 339:819-23; Jinek et al., Elife. (2013) 2:e00471; Lei et al. Cell (2013) 152: 1173-1183; Gilbert et al. Cell (2013) 154:442-51; Lei et al. Elife (2014) 3:e04766; Perez-Pinela et al. Nat Methods (2013) 10: 973-976; Maider et al. Nature Methods (2013) 10, 977-979 which are incorporated by reference. The following U.S. and international patents and patent applications describe the methods of use of CRISPR, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 2014/0068797; and WO 2014/197568, all incorporated by reference in their entirety.

In one aspect, the disclosure provides uses CRISPR-Cas system comprising a Cas9 protein and one or more guide RNAs and optionally repair templates to modify the UBAP1 gene, promoter or non-coding elements associated with the UBAP1 gene. The CRISPR-Cas reagents (e.g. CRISPR-Cas9) used to modify the genes are themselves an aspect of the disclosure. For example, the disclosure includes a guide RNA molecule, e.g., an isolated or non-naturally occurring gRNA molecule, comprising a targeting domain which is complementary with a target domain from the UBAP1 gene, promoter or non-coding elements associated with the UBAP1 gene.

In another aspect, CRISPR-Cas reagents (e.g. CRISPR-Cas9 and guide RNA sequences) are designed and made and used to alter the non-coding sequence of the UBAP1 gene to alter UBAP1 gene expression by targeting, e.g., a promoter, an enhancer, an intron, 3′UTR, and/or polyadenylation signal.

The agent may encode the wild-type sequence of the CRISPR related protein or a variant CRISPR related protein. The agent can include either the wild-type codon usage or codon usage optimized for a particular application, e.g., human codon optimization for human therapeutics. The type II CRISPR Cas9 systems are particularly preferred.

In some embodiments, an agent that corrects the mutation in UBAP1 gene (or enhances expression of the wild-type UBAP1 gene or reduces expression of a mutant UBAP1 gene) uses prime editing as described in Anzalone et al., Nature 576, 149-157 (2019). Prime editing enables the introduction of indels and all 12 base-to-base conversions (both transitions and transversions) without inducing a DNA double-strand break.

In some embodiments, CRISPR/Cas9 multiplexing may be used to target multiple genomic loci wherein 2 or more guide RNAs are expressed as described in CRISPR 101:A Desktop Resource (1^{st} Edition), Addgene, January 2016 which is incorporated by reference in its entirety.

In some embodiments, DNA encoding Cas9 molecules (e.g., eaCas9 molecules) and/or gRNA molecules, are delivered into cells by art-known methods or as described herein. For example, Cas9-encoding and/or gRNA-encoding DNA are delivered, e.g., by vectors (e.g., viral or non-viral vectors), non-vector based methods (e.g., using naked DNA or DNA complexes), or a combination thereof.

In various embodiments, agents to modify the genome of a mammalian subject disclosed herein are delivered via viral vectors. For example, Cas9 and guide RNAs can be present in a single lentiviral transfer vector or separate transfer vectors. Adenoviral delivery of the CRISPR/Cas9 system is described in Holkers et al., Nature Methods (2014), 11(10):1051-1057 which is incorporated by reference in its entirety.

Typically, polynucleotides are delivered to target cells via an expression vector, which includes the regulatory sequences necessary for delivery and expression. In various embodiments, the expression vector includes a promoter (e.g., cytomegalovirus (CMV) promoter), coding and/or non-coding (e.g. 3′-UTR) regions of interest, inteins, transcription termination sequences and/or regulator elements. Tissue specific promoters or inducible promoters may be used. Optionally, the expression vector is a viral vector, such as a lentiviral vector, baculoviral vector, adenoviral vector, or adeno-associated viral vector. Other means of delivery are also appropriate, such as yeast systems, microvesicles, gene guns, nanoparticles-mediated delivery, liposomes, and exosomes.

### Methods of Treatment

In various aspects, the disclosure provides a method of treating Hereditary Spastic Paraplegia (HSP) and other neurological diseases in a mammalian subject (e.g., a human). Other neurological diseases include, but are not limited to, peripheral neuropathies, spinocerebellar ataxias, and motor neuron diseases, specifically including amyotrophic lateral sclerosis (ALS). As used herein, the term “treat,” as well as words related thereto, do not necessarily imply 100% or complete treatment. Rather, there are varying degrees of treatment of which one of ordinary skill in the art recognizes as having a benefit or therapeutic effect. In this respect, the methods of treating a neurological disease, such as HSP, can provide any amount or any level of treatment. Furthermore, the treatment provided by the method of the present disclosure may include treatment of one or more conditions or symptoms or signs of the neurological disease, being treated.

Although more than one route can be used to administer an agent, a particular route can provide a more immediate and more effective reaction than another route. Depending on the circumstances, a pharmaceutical composition comprising the agent is applied or introduced into body cavities, absorbed through the skin or mucous membranes, ingested, inhaled, and/or introduced into circulation. For example, in certain circumstances, it will be desirable to deliver the pharmaceutical composition orally; through injection or infusion by intravenous, intratumoral, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means; by controlled, delayed, sustained or otherwise modified release systems; or by implantation devices. In one aspect, drug exposure can be optimized by maintaining constant drug plasma concentrations over time. Such a steady-state is generally accomplished in clinical settings by continuous drug infusion at doses depending on the drug clearance and the plasma concentration to be sustained. If desired, the composition is administered regionally via intrathecal administration, intracerebral (intra-parenchymal) administration, intracerebroventricular administration, or intraarterial or intravenous administration targeting the region of interest. Alternatively, the UBAP1 peptide, polynucleotide encoding UBAP1, agent that induces expression of an endogenous nucleotide sequence that encodes a UBAP1 peptide, agent that blocks expression of a mutant UBAP1 gene, or agent that corrects the mutation in UBAP1 gene is administered locally via implantation of a matrix, membrane, sponge, or another appropriate material onto which the desired agent has been absorbed or encapsulated. Where an implantation device is used, the device is, in one aspect, implanted into any suitable tissue or organ, and delivery of the desired agent is, for example, via diffusion, timed-release bolus, or continuous administration.

The amount of the agent administered to the subject will depend on the severity disease, physical health of the patient, and the like. Ideally, a therapeutically effective amount is administered which provides a benefit in a clinically relevant period of time.

Agents administered to subjects are typically formulated as a composition comprising one or more pharmaceutically acceptable carriers. “Pharmaceutically (or pharmacologically) acceptable” carriers do not produce allergic or other adverse reactions when administered using routes well-known in the art, and include any and all clinically useful solvents, dispersion media, coatings, antibacterial and antifungal agents, isotonic and absorption delaying agents and the like.

It is contemplated that two or more agents of the present disclosure may be given simultaneously, in the same formulation. It is further contemplated that the two or more agents may be administered in separate formulations administered near in time (i.e., within 30 minutes of each other) or administered separately. Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In some variations one or more agents disclosed herein are administered as a co-therapy in combination with a standard-of-care therapy for treating a neurological disease, such as Hereditary Spastic Paraplegia (HSP). Exemplary standard-of-care HSP therapies include, but are not limited to, muscle relaxants (e.g., Baclofen), botulinum toxin, clonazepam, dantrolene, diazepam, or tizanidine

Additional aspects and details of the disclosure will be apparent from the following examples, which are intended to be illustrative rather than limiting.

### EXAMPLES

The following Examples are merely illustrative and are not intended to limit the scope or content of the invention in any way.

### General Methods

### Subjects and Family Members

All affected cases studied were from non-consanguineous and unrelated families. All families gave written informed consent, and the study protocol was approved by the institutional review board of the participating institutions. Patients were clinically evaluated by neurologists.

### Whole-Exome Sequencing

Whole-exome sequencing was performed in the seven index individuals with autosomal-dominant HSP. The SureSelect Human All Exon Kit (Agilent) was used for in-solution enrichment, and the HiSeq 2500 instrument (Illumina) was used to produce 100 bp paired-end sequence reads. The Burrows-Wheeler aligner and Freebayes were used for sequence alignment and variant calling. Exome data were uploaded into the GENESIS software and analyzed with strict filtering approach for heterozygous variants. Sanger sequencing confirmed segregation of the loss-of-function variants detected by whole-exome sequencing in the seven HSP families.

### Western Blot

Fibroblasts from affected individual from family 1 were cultured and cell lysates were collected for Western blot analysis. The following antibodies were used to detect UBAP1 and GAPDH: Rabbit polyclonal anti-UBAP1 antibody (Abcam) and mouse monoclonal GAPDH antibody (Santa Cruz).

### Reverse Transcription Polymerase Chain Reaction (RT-PCR) and Sanger Sequencing

Total RNA was isolated from patient's fibroblast using the RNeasy Plus Mini Kit (Qiagen). cDNA was synthesized from purified RNA with the SuperScript™ III First-Strand Synthesis SuperMix (ThermoFisher). Primers were used to amplify mutation site including exons 4 and 5 and flanking intronic region: 5′-CCACAATGCCACCTCCTAT-3′ (SEQ ID NO: 4) (forward) and 5′-AGAATAGGCCTGGGGACA-3 (SEQ ID NO: 5) (reverse). Polymerase chain reaction (PCR) was performed with Platinum Taq (ThermoFisher) and PCR products were purified with the PCR purification kit (Qiagen). Sanger sequencing was performed by Eurofins Genomics, and trace files were analyzed with the Sequencher software.

### Plasmids Transfection and Immunofluorescence

Plasmid encoding open-reading frame of UBAP1 transcript 1 (NM_016525.4) fused with a 3×-HA tag in the N-terminus end of the protein was obtained from Genecopoeia. Plasmid encoding VPS28 transcript (NM_016208.3) fused to a Myc tag at the C-terminus region was also obtained from Genecopoeia. Site-direct-mutagenesis was performed to generate frameshift mutation (HA-FS-UBAP1) corresponding to variant c.361dupC (p.Leu121Profs*18). One base insertion was introduced by PCR using the Q5 Site-Direct Mutagenesis kit (NEB). U2OS cells were transfected with Lipofectamine 3000. Cells were plated on cover slips and immonostained with anti-HA and anti-myc (Cell signaling) and fluorescent antibodies, Alexa fluor 488 and Alexa fluor 555 (Invitrogen). Cells were imaged by confocal microscopy (LSM710).

### Co-Immunoprecipitations

HEK293T cells were transiently transfected with VPS28-Myc and HA-WT-UBAP1 or HA-Fs-UBAP1. After 24 hrs cells were harvested in IP lysis/wash buffer (Thermofisher) and incubated on ice for 5 minutes. Cell debris was removed by centrifugation (13,0000 g). Co-immunoprecipitation was performed according to manufacturer's protocol. Briefly, Antibodies (HA, Myc and IgG) were incubated with magnetic Dynabeads (ThermoFisher) for 10 minutes and cell lysates were incubated with the dynabeads-antibody complex for 10 minutes. Dynabeads were washed three times with washing buffer and eluted with elution buffer.

### Zebrafish Husbandry

Experiments were carried out using transgenic stain Tg(olig2::DsRed).¹⁵ Adults were kept on a 14-h light/10-h dark cycle at 28° C. Embryos were collected from natural crosses after removing a divider at the beginning of the light cycle. Embryos were raised in Petri dishes in the system water at 28° C. under standard conditions. All experiments were conducted in accordance with University of Miami Institutional Animal Care and Use Committee guidelines. For live imaging embryos were anesthetized at 48 hpf with 0.02% tricaine methanesulfonate (Sigma).

sgRNA Design and Synthesis

sgRNAs were chosen among top targets with NGG PAM sites generated by the CHOPCHOP software (http://chopchop.cbu.uib.no/) with zero predicted off-targets with at least three mismatches in the 20-mer. The target exons chosen were exon 4 and exon 7 (two guides per exon). sgRNAs were generated by the oligonucleotide assembly method as described in Varshney et al.¹⁶ RNAs were synthesized using the HiScribe™ T7 Quick High Yield RNA Synthesis Kit (New England Biolabs) with an incubation time of 12 h for the in vitro transcription reaction. RNAs were purified with the RNA Clean & Concentrator™-5 kit (Zymo Research) and eluted with 15 μl water and diluted to working concentrations ˜200 pg/μl.

hRNA Rescue Synthesis

hRNA was synthesized from plasmid encoding wild-type and truncated mutant UBAP1 (family 4, p.Leu121Profs*18) using mMessage mMachine™ T7 Ultra kit (Invitrogen).

### Microinjections

Microinjections were performed into embryos at one-cell stage. Cas9 protein (PNA Bio) and pooled sgRNA were mixed with 1% Fast Green dye (Sigma) and incubated for 5 min at 37 degrees Celsius. For the rescue injections, mixtures were supplemented with either wild-type or mutant synthesized human RNA (hRNA). Approximately 1.5 nL of active sgRNA-Cas9 ribonucleoprotein complex plus hRNA were injected per embryo into the cell. The final amounts injected per embryo approximately were: 350 pg of Cas9 protein; 150 pg of sgRNA pool, 50 pg of hRNA. At least three independent injection experiments were performed with spawns from different founder fish.

### In Vivo Imaging of Motor Neurons

Motoneuron outgrowth was assayed at 48 h.p.f. Live fish were dechorionated by tweezers and anesthetized with tricaine methanesulfonate, embedded in 1% low-melting point agarose and imaged using a Leica confocal microscope with a 20× air lens. 1-μm z stacks were imaged between myotome segments 6 and 13, and the motoneuron morphology was evaluated for its normal shape and outgrowth trajectory.¹⁷ Images were processed with Fiji software (ImageJ). LUT:edges was used to generate the figure.

### CRISPR Efficiency Testing by Fragment Analysis

Embryos were euthanized and DNA was extracted using 50 mM NaOH digestion at 95 degrees Celsius for 20 min. DNA then was used to run Fluorescent PCR as described in Varshney et al. ¹⁶ The reaction products were run on a Genetic Analyser 3130×1 using POP-7 polymer and analyzed for the disruption of the wild-type peaks as described in Carrington et al.¹⁸

### Statistical Analysis

Images from three separate experiments were blindly evaluated for qualitative inclusion into either “normal” group or “affected” group. The normal group was assigned to images with motor axons shaped into normal hooks as in images of uninjected controls. The affected group was assigned to images with any amount of drastically misshaped or truncated axons. The Fisher exact test was performed for these two groups comparing the wild-type rescue injected group with the mutant rescue group. Differences in the number of observations were considered significant at p≤0.05. To quantify the motoneuron axon length the Simple Neurite Tracer plugin in Fiji was used for tracing.¹⁹ Four axons per sample embryo from the same area closer to the yolk were traced, and the statistical comparison was performed by using one-tailed Student's t test with the p-value considered significant under p≤0.005.

### Example 1: Identification of an Autosomal-Dominant Gene for Hereditary Spastic Paraplegia (HSP)

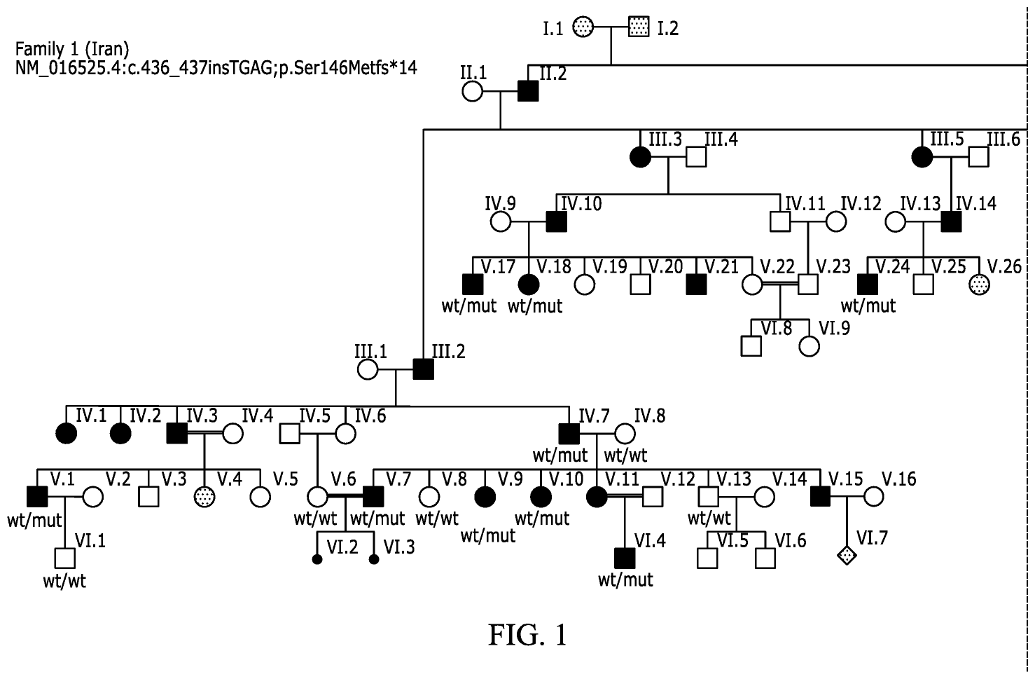

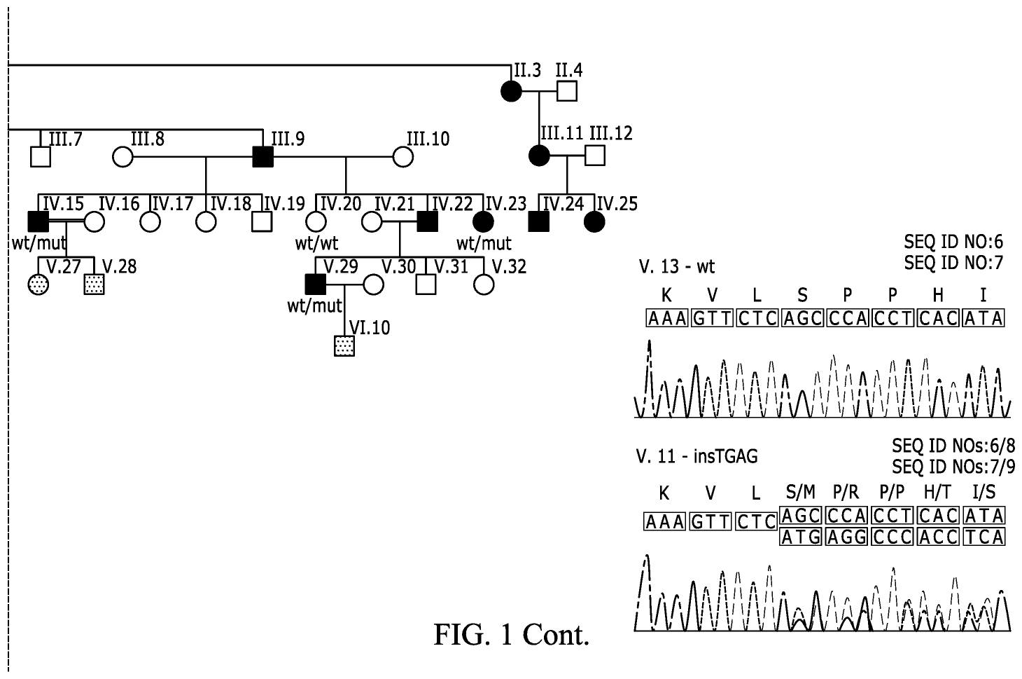

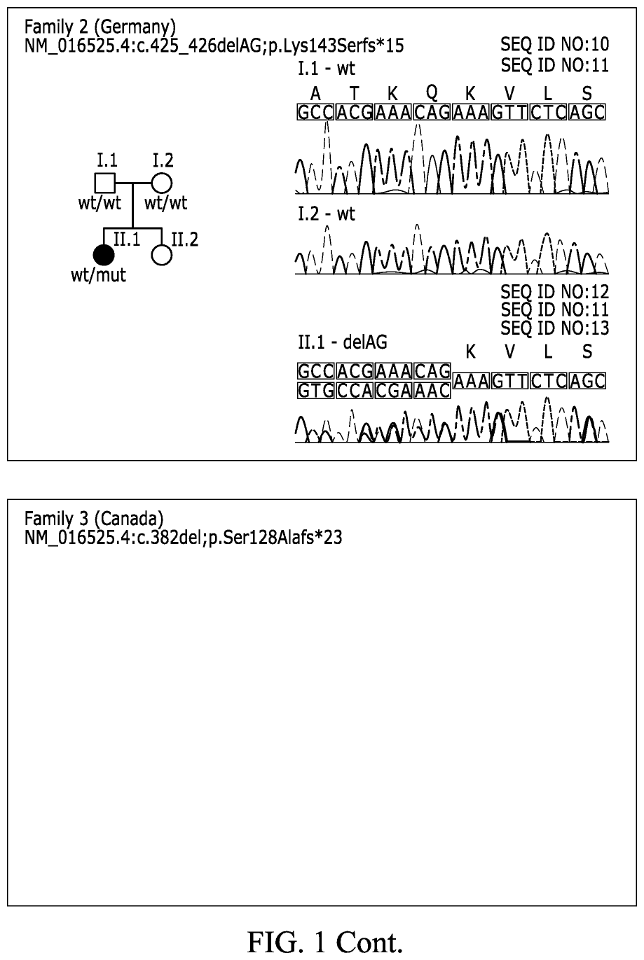

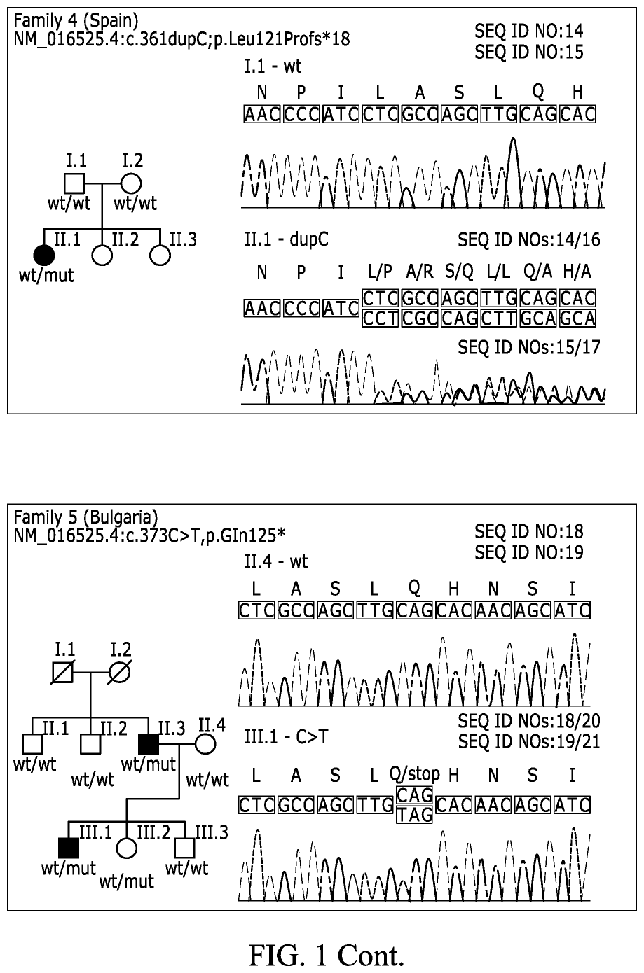

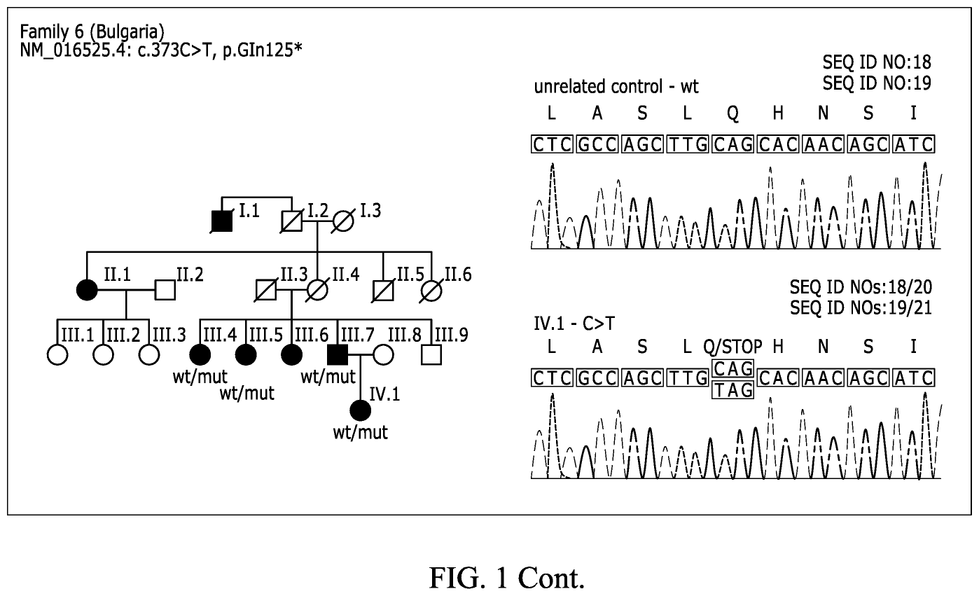

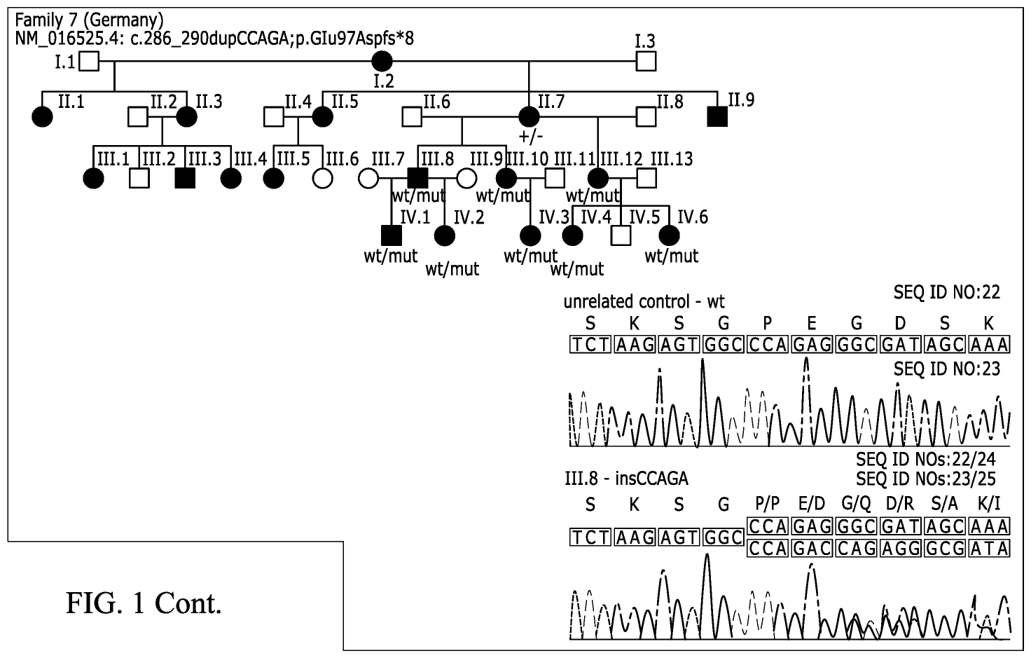

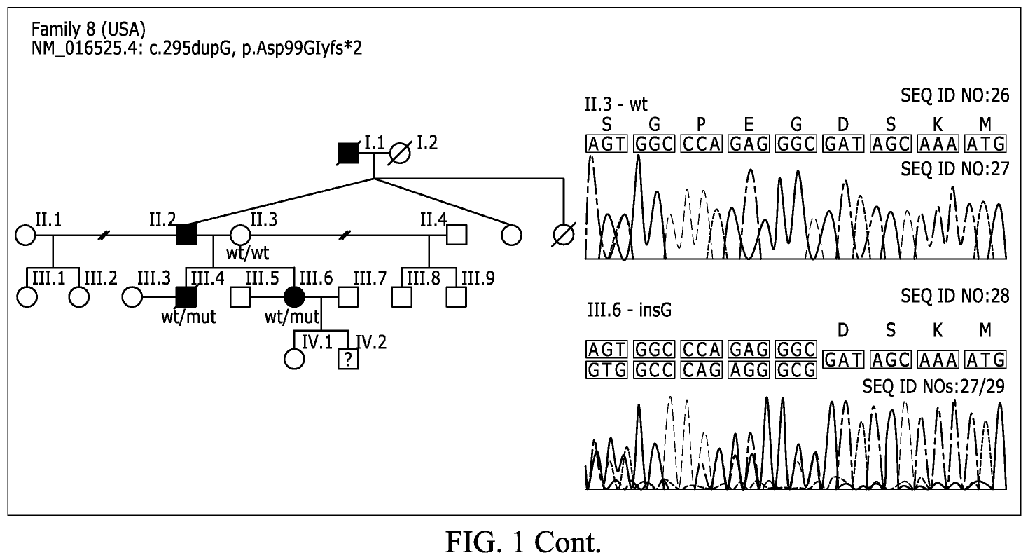

To further close the diagnostic gap in HSP, a highly diverse sample of ten families from six countries (Iran:1, USA:1, Germany:4, Canada:1, Bulgaria:2, Spain:1) was collected. All participating patients gave informed consent prior to initiating this study in agreement with each institutional review board. In one family of Persian origin, family 1, it was possible to genetically ascertain a total of 14 affected individuals from three generations (FIG. 1). Sequencing of an HSP gene panel and CNV analysis at the SPG4 locus were unremarkable. Subsequently, WES was performed in two affected individuals (V.1 and V.15). Bioinformatics analysis of the sequencing data used standard tools, including BWA aligner⁶, FreeBayes⁶, GATK⁸, and GENESIS⁹. Only non-synonymous variants with a minor allele frequency of less than 0.0001 in gnomAD and an in-house Iranian variant database of 1,500 exomes (BayanGene) were further considered. Two heterozygous variants remained in the genes SVEP1 (chr9:113137668; rs373655861; p.Thr3527Met, hg19) and UBAP1 (chr9:34241270; NM_016525.4: c.436_437insTGAG, p.Ser146Metfs*14), respectively. The SVEP1 variant was ruled out by segregation studies of the entire pedigree applying Sanger sequencing. Thus, after confirmation of complete segregation, the truncating frameshift variant in Ubiquitin Associated Protein 1 (UBAP1) was considered as the causative allele in family 1 (FIG. 1). This frameshift variant was not present in ExAC, gnomAD, GENESIS, nor in 1,500 Iranian genomes. It is predicted to truncate the protein at residue 158 out of 502 amino acids (NM_016525.4). By including the Sanger confirmed affected and unaffected participants (FIG. 1), parametric two-point linkage analysis using LINKAGE program was performed, which rendered a two-point LOD score of 8.25 at the position of the UBAP1 insertion.

### Example 2: Identification of UBAP1 Variants

A search was then conducted for additional families with UBAP1 variants in the GENESIS database, which contains more than 3,000 exomes and genomes from patients with HSP and related disorders.⁹ By filtering for non-synonymous and truncating variants under an autosomal-dominant model with minor allele frequency in gnomAD<0.0001 and a minimum sequencing depth of ten reads, seven additional HSP families were identified, all carrying truncating variants in UBAP1 (Table 1). In addition, predictively truncating UBAP1 variants were prioritized in two families (9 and 10) who underwent diagnostic exome sequencing at the University of Tuebingen. The detection of truncating alleles in all families is especially remarkable considering the almost complete constraint of UBAP1 for loss-of-function (truncating) variation in the ˜120,000 chromosomes in both ExAC and gnomAD, pLi=0.95 and 0.92 respectively.¹⁰ The probability of significant enrichment of truncating variations in UBAP1 was calculated in the HSP dataset compared to ExAC. In the Genesis dataset 7 such variants were identified in a cohort of 567 HSP samples versus 0 truncating variants in 60,000 ExAC samples (P=6.187e-15 by Fisher test. Odds ratio=Inf.). Five truncating variants were reported in the ˜246,000 chromosomes in gnomAD, but none fell within the specific gene region containing the variants reported in this study.

All additional variants and their segregation with disease in the additional families were confirmed by Sanger sequencing. The identified variants were as follows based on transcript NM_016525.4 (Table 1 and FIG. 1): families 2 and 10 from Germany, c.426_427delGA, p.Lys143Serfs*15; family 3 from Canada, c.382del, p.Ser128Alafs*23; family 4 from Spain, c.361dupC, p.Leu121Profs*18; family 5 and 6 from Bulgaria (Roma ethnicity), c.373C>T, p.Gln125*; family 7 from Germany, c.286_290dupCCAGA, p.Glu97Aspfs*8; family 8 from United States, c.295dupG, p.Asp99Glyfs*2 and family 9 from Germany, c.1091delC, p.Pro364Leufs*50.

TABLE 1
Detailed genomic locations of detected pathogenic variants.
Isoform 1 is referred to throughout the text.

| | | Isoform 1 | | Isoform 4 |
|---|---|---|---|---|
| | | (NM_016525.4) | | (NM_001171201.1) |
| Family | Genome assembly | Expressed in neurons | | Canonical according to NCBI |
| ID | (hg19) | cDNA | protein | protein |
| 1 | chr9:34241459- | c.436_437insTGAG | p.Ser146Metfs*14 | p.Ser210Metfs*14 |
| | 34241460 | | | |
| 2 | chr9:34241449- | c.426_427delGA | p.Lys143Serfs*15 | p.Lys207Serfs*15 |
| | 34241450 | | | |
| 3 | chr9:34241405- | c.382del | p.Ser128Alafs*23 | p.Ser192Alafs*23 |
| | 34241405 | | | |
| 4 | chr9:34241384- | c.361dupC | p.Leu121Profs*18 | p.Leu185Profs*18 |
| | 34241384 | | | |
| 5 | chr9:34241396- | c.373C > T | p.Gln125* | p.Gln161* |
| | 34241396 | | | |
| 6 | chr9:34241396- | c.373C > T | p.Gln125* | p.Gln161* |
| | 34241396 | | | |
| 7 | chr9:34241309- | c.286_290dupCCAGA | p.Glu97Aspfs*8 | p.Glu161Aspfs*8 |
| | 34241313 | | | |
| 8 | chr9:34241318- | c.295dupG | p.Asp99Glyfs*2 | p.Asp163Glyfs*2 |
| | 34241318 | | | |
| 9 | chr9:34249784- | c.1091delC | p.Pro364Leufs*50 | p.Pro428Leufs*50 |
| | 34249784 | | | |
| 10 | chr9:34241449- | c.426_427delGA | p.Lys143Serfs*15 | p.Lys207Serfs*15 |
| | 34241450 | | | |

In two of the families, Family 2 and Family 4, a de novo occurrence of the truncating variant was confirmed (FIG. 1). Families 5 and 6 were of self-declared Bulgarian Roma ethnicity and carried the same variant p.Gln125*, albeit the two index participants are from reportedly unrelated families. Further studies will be necessary to evaluate the prevalence of this allele in European Roma population and in Gypsy HSP patients.

The first manifesting symptom in all 30 UBAP1 mutation carriers from 10 families with detailed clinical data was a progressive spastic gait disorders with a median age at onset of 8 years (interquartile range 4-9 years, oldest onset age 26 years, one asymptomatic mutation carrier at age 14 (F5-III.2) (detailed clinical information in supplementary table 1)). At the time of examination (median disease duration 28 years, interquartile range 15-36 years), lower limb spastic paraparesis was still the most prominent clinical feature in all affected mutation carriers with brisk lower limb tendon reflexes (all, including asymptomatic carrier F5-III.2) and extensor plantar response in all but the youngest affected (F7-IV.6). While brisk tendon reflexes of the upper limbs were frequently present (26 of 30, 87%), significant upper limb spasticity was seen only in a single case (1/30, 3%, F4-II.1), consistent with a length dependent axonopathy of the corticospinal tract. Urinary urgency was reported in some cases (11 of 30, 37%), sensory deficits were absent or mild and there was no evidence of peripheral neuropathy. In the majority of families (8/10, 80%), no additional signs or symptoms indicating affection of neuronal systems other than the corticospinal tract were seen, and the disease accordingly classified as pure HSP. In family 7, however, 7 out of 9 family members had features of cerebellar involvement (saccadic pursuit, gaze evoked nystagmus, dysmetric saccades, limb ataxia), features also present in family 9 (F9-II.1), indicating that the cerebellum is vulnerable to UBAP1 dysfunction at least in some cases.

Overall, truncating UBAP1 mutations are associated with a predominantly pure early-onset HSP phenotype; cerebellar involvement seems to be clustered in families and was observed in 2/10 families. Although there is thus little phenotypic variability in terms of system involvement across UBAP1 families, there seems to be quite some variability regarding the progression rate with rather rapid disease progression and early wheelchair dependency, e.g., in families 2, 6, and 7 on the one hand and almost non-progressive disease in family 9 (F9-II.1 still able to run and walk unlimited distances after 38 years of disease duration). Both intrafamilial as well as interfamilial variability are common or even the norm in HSP.⁴ Careful clinical evaluation of additional UBAP1 families will be needed to fully understand the phenotypic spectrum associated with UBAP1 mutations.

### Example 3: Investigating the Effects of the Truncated Mutant UBAP1 Protein

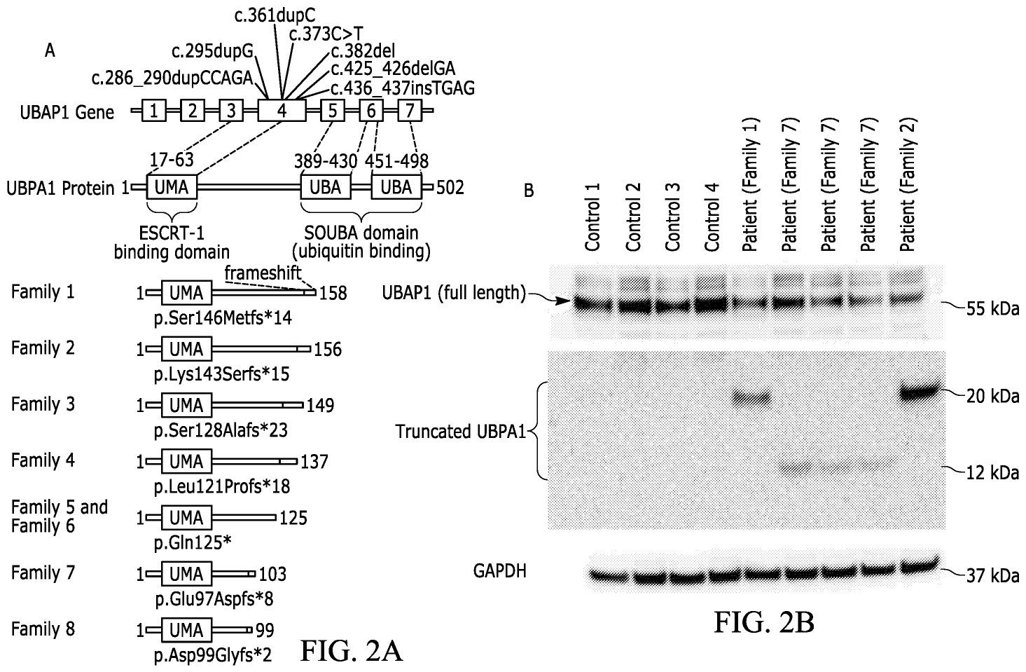

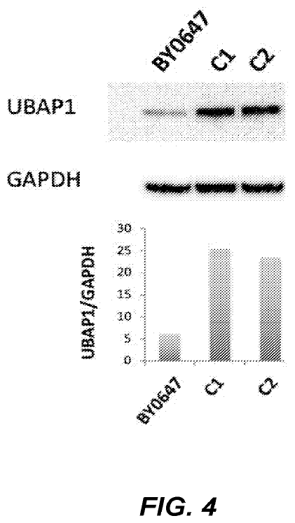

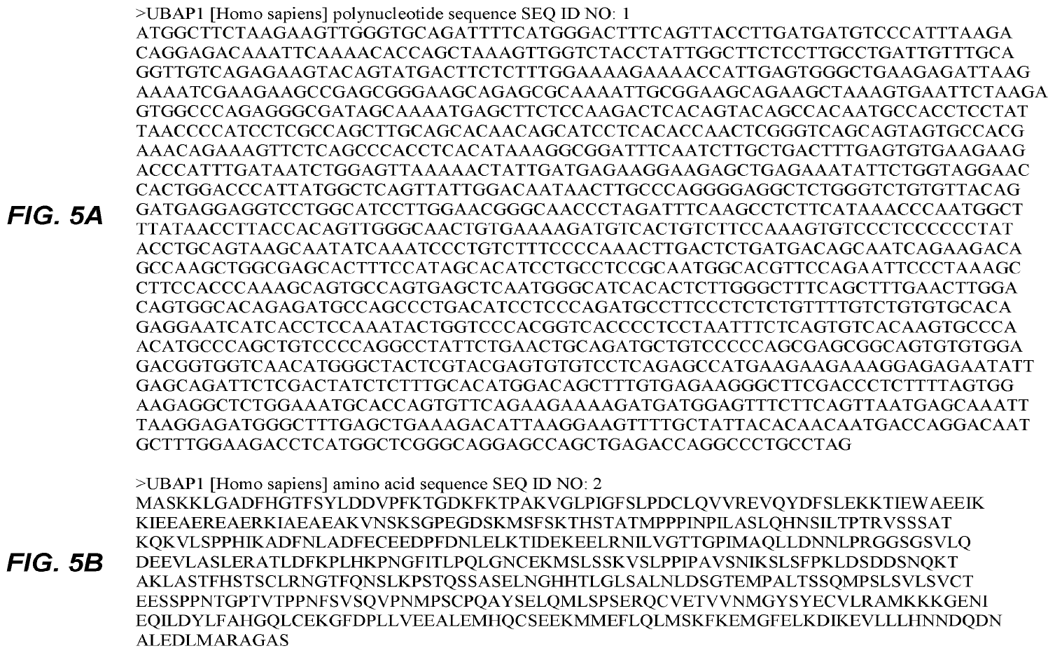

UBAP1 is a member of the ESCRT-I complex and a regulator of vesicular trafficking processes, it binds to ubiquitinated cargo proteins, and is essential to sort endocytic ubiquitinated cargos into multivesicular bodies (MVBs).¹¹ It also plays an important role in proteasomal degradation of ubiquitinated cell-surface proteins, including EGFR (Epidermal growth factor receptor) and BST2 (Bone Marrow Stromal Cell Antigen 2).¹¹ UBAP1 protein has two main domains: The UMA (UBAP1-MVB12-associated) domain in the N-terminal region (17-63 aa), which mediates the association with the ESCRT-I complex and a solenoid of overlapping ubiquitin-associate UBAs (SOUBA) domain in the C-terminal region (389-498 aa). ^{11,12} Both domains allow UBAP1 to act as a molecular bridge connecting the endosomal trafficking pathways to the ubiquitination machinery. To decipher the pathophysiology of UBAP1 in HSP it was noted that all but one of the identified changes fall within a circumscript area of the protein, between Asp99 and Ser 146, with the change in family 9 at Pro364 being the only outlier (NM_016525.4). Interestingly, disease progression in this family is dramatically slower than in the other families with almost stationary disease over decades (see above), pointing towards a possible genotype-phenotype correlation. Yet, all changes preserve the UMA domain, but cause a loss of the SOUBA domain.¹² It has been shown that mutagenesis of the SOUBA domain in UBAP1 strongly reduces its interaction with ubiquitinated proteins (FIG. 2).¹² To determine whether the observed truncating variants will lead to nonsense-medicated mRNA decay and haploinsufficiency, both the RNA and protein expression of mutant alleles were evaluated. RT-PCR was performed on RNA extracted from patient's fibroblasts and Sanger sequenced. Surprisingly, the c.436_437insTGAG was detected in the patient's cDNA, indicating escape of nonsense-medicated mRNA decay (FIG. 4). Next, Western blot analysis was performed to evaluate the levels of both wild-type and potential truncated mutant UBAP1 protein. Total protein extracts were probed with an antibody raised against the N-terminus region of UBAP1 (amino acids 25-75), a part of the protein preserved in mutant UBAP1 proteins. The protein levels measured in patients were compared with four control fibroblasts and normalized to GAPDH levels. Western blots showed decreased protein levels of full-length UBAP1 in patient fibroblasts; in addition, the truncated protein was detected (FIG. 2). The reduced levels of the full-length protein in patient's fibroblast compared to controls along with the presence of the truncated protein could potentially lead to haploinsufficiency and/or dominant negative effect.

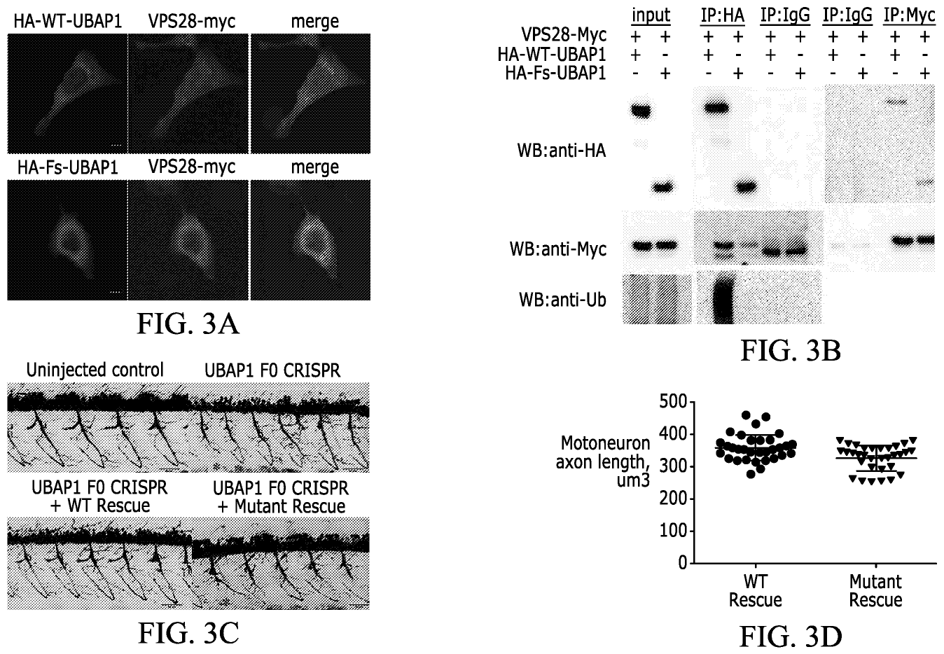

To evaluate the effects of the truncated protein, site-direct mutagenesis was performed and generated a plasmid encoding the truncated protein fused to an HA tag at the N-terminus region. U2OS cells were co-transfected with either wild-type (HA-WT-UBAP1) or a truncated mutant (HA-Fs-UBAP1; p.Leu121Profs*18) together with its known binding partner VPS28-Myc. Both wild-type and truncated mutant co-localize with VPS28 (FIG. 3A). This suggests that interaction with ESCRT-I complex is preserved; however, the lack of the SOUBA domain, essential for binding of ubiquitin, would be detrimental. Interestingly, overexpression of truncated protein containing the UMA domain has been shown to result in a dominant negative effect by inhibiting HIV-1 budding.¹² It is thus possible that expression of the truncated protein in patients could cause a dominant negative effect due to arrest of ESCRT-complex without acquiring the ubiquitinated protein cargo.

A co-immunoprecipitation (Co-IP) assay was performed to confirm the interaction between HA-Fs-UBAP1 and VPS28-Myc. HEK293T cells were co-transfected with VPS28-Myc and with either HA-WT-UBAP1 or HA-Fs-UBAP1 and immunoprecipitated with an anti-HA or anti-Myc antibodies and analyzed by Western blots. The results show that both wild-type and truncated UBAP1 co-immunoprecipitated with VPS28 confirming protein-protein interaction (FIG. 3B). However, ubiquitinated proteins were co-immunoprecipitated with the HA-WT-UBAP1, but not with HA-Fs-UBAP1. It has previously been described that siRNA depletion of UBAP1 in HeLa cells causes clustering of early endosomes, accumulation of ubiquitinated proteins and enlargement and clustering of LAMP1-positive late endosomes and lysosomes.¹³ In fibroblasts of patients carrying UBAP1 mutations, however, none of these changes could be observed, not even after exposing cells to stress conditions. It therefore appears unlikely that loss of one UBAP1 allele results in gross failure of multivesicular body sorting.

To investigate the effects of the truncated protein in vivo, a zebrafish model with UBAP1 knockdown was generated. A transgenic fish with fluorescently labeled motoneuron Tg(olig2::DsRed) was used.¹⁴ Embryos were injected with CRISPR Cas9 and sgRNAs against UBAP1 supplemented with either human RNA rescue of wild-type or truncated mutant UBAP1. At 48 hours post-fertilization (hpf) embryos were in vivo imaged with a confocal microscope. Significantly more truncated and misshaped axons were observed in the mutant rescue compared to the wild-type rescued embryos (FIG. 3C). Motoneuron axons length in the truncated mutant were significantly shorter compared to wild-type. This result supports the pathogenic effects of the truncated protein in vivo.

In summary, disclosed herein is strong genetic evidence for truncating mutations in UBAP1 to cause a relatively frequent form of HSP. UBAP1 mutations were identified in a large Iranian kindred, as well as in seven additional families with different ancestral backgrounds from Bulgarian Roma, European descent North American, German, Spain, and Quebec. All available patients in these families carried the respective mutation in UBAP1, albeit UBAP1 has a strong loss-of-function constrain in the 60,000 individuals studied in the ExAC dataset. In two families, it was also possible to show a de novo occurrence of the variants. In the dataset of 567 dominant HSP families, UBAP1 accounts for 1.2% of cases.

### REFERENCES

1. Fink J K: Hereditary Spastic Paraplegia Overview. In: GeneReviews®. Edited by Adam M P, Ardinger H H, Pagon R A, Wallace S E, Bean L J H, Stephens K, Amemiya A. Seattle (WA); 1993. [symbol]
2. Fink J K: Hereditary spastic paraplegia: clinico-pathologic features and emerging molecular mechanisms. Acta neuropathologica 2013, 126(3):307-328. [symbol]
3. Parodi L, Fenu S, Stevanin G, Dun A: Hereditary spastic paraplegia: More than an upper motor neuron disease. Rev Neurol (Paris) 2017, 173(5):352-360.
4. Schüle R, Weithoff S, Martus P, Karle K N, Otto S, Klebe S, Klimpe S, Gallenmüller C, Kurzwelly D, Henkel D et al: Hereditary spastic paraplegia: Clinicogenetics lessons from 608 patients. Annals of Neurology 2016, 79(4). [symbol]
5. Bis-Brewer D M and Züchner S: Perspectives on the Genomics of HSP Beyond Mendelian Inheritance. Frontiers in Neurology 2018, 9(958). [symbol]
6. Li H, Durbin R: Fast and accurate short read alignment with Burrows-Wheeler transform. Bioinformatics 2009, 25(14):1754-1760. [symbol]
7. Garrison E, Marth G: Haplotype-based variant detection from short-read sequencing. arXiv preprint arXiv:1207.3907 [symbol]
8. McKenna A, Hanna M, Banks E, Sivachenko A, Cibulskis K, Kernytsky A, Garimella K, Altshuler D, Gabriel S, Daly M et al: The Genome Analysis Toolkit: a MapReduce framework for analyzing next-generation DNA sequencing data. Genome research 2010, 20(9):1297-1303. [symbol]
9. Gonzalez M, Falk M J, Gai X, Postrel R, Schule R, Zuchner S: Innovative genomic collaboration using the GENESIS (GEM.app) platform. Human mutation 2015, 36(10):950-956. [symbol]
10. Lek M, Karczewski K J, Minikel E V, Samocha K E, Banks E, Fennell T, O'Donnell-Luria A H, Ware J S, Hill A J, Cummings B B et al: Analysis of protein-coding genetic variation in 60,706 humans. Nature 2016, 536(7616):285-291. [symbol]
11. Stefani F, Zhang L, Taylor S, Donovan J, Rollinson S, Doyotte A, Brownhill K, Bennion J, Pickering-Brown S, Woodman P: UBAP1 is a component of an endosome-specific ESCRT-I complex that is essential for MVB sorting. Curr Biol 2011, 21(14):1245-1250. [symbol]
12. Agromayor M, Soler N, Caballe A, Kueck T, Freund S M, Allen M D, Bycroft M, Perisic O, Ye Y, McDonald B et al: The UBAP1 subunit of ESCRT-I interacts with ubiquitin via aSOUBA domain. Structure 2012, 20(3):414-428. [symbol]
13. Stefani F, Zhang L, Taylor S, Donovan J, Rollinson S, Doyotte A, Brownhill K, Bennion J, Pickering-Brown S, Woodman P: UBAP1 is a component of an endosome-specific ESCRT-I complex that is essential for MVB sorting. Curr Biol 2011, 21(14):1245-50.
14. Kucenas S, Snell H, Appel B. nkx2.2a promotes specification and differentiation of a myelinating subset of oligodendrocyte lineage cells in zebrafish. Neuron Glia Biol 2008, 4 (2):71-81.
15. Kucenas, S., Snell, H., and Appel, B. (2008). nkx2.2a promotes specification and differentiation of a myelinating subset of oligodendrocyte lineage cells in zebrafish. Neuron Glia Biol. 4, 71-81.
16. Varshney, G. K., Pei, W., LaFave, M.C., Idol, J., Xu, L., Gallardo, V., Carrington, B., Bishop, K., Jones, M., Li, M., et al. (2015). High-throughput gene targeting and phenotyping in zebrafish using CRISPR/Cas9. Genome Research 25, 1030-1042.
17. Myers, P. Z., Eisen, J. S., and Westerfield, M. (1986). Development and axonal outgrowth of identified motoneurons in the zebrafish. Journal of Neuroscience 6, 2278-2289.
18. Carrington, B., Varshney, G. K., Burgess, S. M., and Sood, R. (2015). CRISPR-STAT: an easy and reliable. PCR-based method to evaluate target-specific sgRNA activity. Nucleic Acids Res. 43, e157-e157.
19. Abrams, A. J., Hufnagel, R. B., Rebelo, A., Zanna, C., Patel, N., Gonzalez, M.A., Campeanu, I. J., Griffin, L. B., Groenewald, S., Strickland, A. V., et al. (2015). Mutations in SLC25A46, encoding a UGO1-like protein, cause an optic atrophy spectrum disorder. Nat. Genet. 47, 926-932.

1. A method of treating neurological disease in a mammalian subject, the method comprising detecting the presence of a truncating mutation in a Ubiquitin Associated Protein 1 (UBAP1) gene in a biological sample from the subject; and
administering to the subject a composition that comprises an agent selected from the group consisting of a UBAP1 peptide; polynucleotide that encodes a UBAP1 peptide; an agent that induces expression of an endogenous nucleotide sequence that encodes a UBAP1 peptide; an agent that blocks expression of a mutant UBAP1 gene; and an agent that corrects the mutation in UBAP1 gene.

2. The method of claim 1, wherein the neurological disease is Hereditary spastic paraplegia (HSP).

3. The method of claim 1, wherein the neurological disease is peripheral neuropathy, spinocerebellar ataxias, motor neuron disease or amyotrophic lateral sclerosis (ALS).

4. The method of claim 1, wherein the agent comprises a UBAP1 peptide, an expression vector comprising a polynucleotide encoding the UBAP1 peptide, a UBAP1 antisense oligonucleotide, a CRISPR Cas9 protein and one or more guide RNA molecules, or combinations thereof.

5. The method of claim 1, wherein the truncating mutation in UBAP1 is c.436_437insTGAG, c.426_427delGA, c.382del, c.361dupC, c.373C>T, c.373C>T, c.286_290dupCCAGA, c.295dupG, c.1091delC, c.426_427delGA, or combinations thereof.

6. The method of claim 2, wherein the agent comprises a UBAP1 peptide, an expression vector comprising a polynucleotide encoding the UBAP1 peptide, a UBAP1 antisense oligonucleotide, a CRISPR Cas9 protein and one or more guide RNA molecules, or combinations thereof.

7. The method of claim 3, wherein the agent comprises a UBAP1 peptide, an expression vector comprising a polynucleotide encoding the UBAP1 peptide, a UBAP1 antisense oligonucleotide, a CRISPR Cas9 protein and one or more guide RNA molecules, or combinations thereof.

8. The method of claim 2, wherein the truncating mutation in UBAP1 is c.436_437insTGAG, c.426_427delGA, c.382del, c.361dupC, c.373C>T, c.373C>T, c.286_290dupCCAGA, c.295dupG, c.1091delC, c.426_427delGA, or combinations thereof.

9. The method of claim 3, wherein the truncating mutation in UBAP1 is c.436_437insTGAG, c.426_427delGA, c.382del, c.361dupC, c.373C>T, c.373C>T, c.286_290dupCCAGA, c.295dupG, c.1091delC, c.426_427delGA, or combinations thereof.

10. The method of claim 4, wherein the truncating mutation in UBAP1 is c.436_437insTGAG, c.426_427delGA, c.382del, c.361dupC, c.373C>T, c.373C>T, c.286_290dupCCAGA, c.295dupG, c.1091delC, c.426_427delGA, or combinations thereof.
